(19) United States
(12) Patent Application Publication
(10) Pub. No.: US 2019/0254563 A1
(43) Pub. Date: Aug. 22, 2019

(54) ENDOSCOPE INSERTION SHAPE OBSERVATION APPARATUS

(71) Applicant: OLYMPUS CORPORATION, Tokyo (JP)

(72) Inventors: Takechiyo NAKAMITSU, Hachioji-shi (JP); Kensuke MIYAKE, Hamura-shi (JP); Akira MURATA, Inagi-shi (JP)

(73) Assignee: OLYMPUS CORPORATION, Tokyo (JP)

(21) Appl. No.: 16/401,425

(22) Filed: May 2, 2019

Related U.S. Application Data
(63) Continuation of application No. PCT/JP2017/035875, filed on Oct. 2, 2017.

(30) Foreign Application Priority Data
Dec. 19, 2016 (JP) 2016-245633

(51) Int. Cl. A61B 5/06; A61B 1/00; A61B 5/00

(52) U.S. Cl. CPC A61B 5/062; A61B 5/7445; A61B 5/066; A61B 1/0005

(57) ABSTRACT

An endoscope insertion shape observation apparatus includes an insertion section configured to be inserted into a subject, and a processor that detects an insertion shape of the insertion section, generates an insertion shape image representing the insertion shape, generates a body outer shape image representing a body outer shape of the subject, and performs display control to simultaneously display the insertion shape image and the body outer shape image in a positional relationship corresponding to a positional relationship, in a body cavity of the subject, of the insertion section on a display screen. The display control also switches between display or non-display of the body outer shape image or a type of the body outer shape image depending on an insertion position, in the body cavity of the subject, of the insertion section.

### CROSS REFERENCE TO RELATED APPLICATION

This application is a continuation application of PCT/JP2017/035875 filed on Oct. 2, 2017 and claims benefit of Japanese Application No. 2016-245633 filed in Japan on Dec. 19, 2016, the entire contents of which are incorporated herein by this reference.

### BACKGROUND

### 1. Field of the Invention

The present embodiments relate to an endoscope insertion shape observation apparatus that observes an inserted state of an endoscope.

### 2. Description of the Related Art

Conventionally, an endoscope apparatus has been widely used in a medical field. The endoscope apparatus is a medical apparatus including an elongated insertion section having flexibility, and an operator can observe an inside of a subject by inserting the insertion section into the subject. An endoscope image in the subject picked up by an endoscope can be displayed on a monitor. However, it cannot be known how the endoscope insertion section has been inserted into the subject from the endoscope image.

Therefore, an endoscope insertion shape observation apparatus is used as an apparatus capable of knowing an inserted state of an endoscope at the time of insertion of the endoscope.

A conventional endoscope insertion shape observation apparatus displays an insertion shape image indicating in what shape an endoscope insertion section has been inserted in a body cavity and does not indicate into which position in the body cavity the endoscope insertion section has been inserted. An operator who operates an endoscope uses images of an inserted state of the endoscope insertion section in the body cavity by the insertion shape image displayed on a monitor, an operation feeling of a hand at the time of the insertion of the endoscope insertion section, and/or an endoscope image at the time of the insertion, for example.

### SUMMARY

An endoscope insertion shape observation apparatus according to an exemplary embodiment includes an insertion section configured to be inserted into a subject, and a processor, in which the processor detects an insertion shape of the insertion section, generates an insertion shape image representing the insertion shape, generates a body outer shape image representing a body outer shape of the subject, and performs display control to simultaneously display the insertion shape image and the body outer shape image in a positional relationship corresponding to a positional relationship, in a body cavity of the subject, of the insertion section on a display screen of a display section, and the display control switches between display or non-display of the body outer shape image or a type of the body outer shape image depending on an insertion position, in the body cavity of the subject, of the insertion section.

An endoscope insertion shape observation apparatus according to another exemplary embodiment includes an insertion section configured to be inserted into a subject, and a processor, in which the processor detects an insertion shape of the insertion section, generates an insertion shape image representing the insertion shape, generates a body outer shape image representing a body outer shape of the subject, and performs display control to simultaneously display the insertion shape image and the body outer shape image in a positional relationship corresponding to a positional relationship, in a body cavity of the subject, of the insertion section on a display screen of a display section, and the display control switches between display or non-display of the body outer shape image or a type of the body outer shape image depending on whether or not the insertion section has a predetermined shape in the body cavity of the subject.

### BRIEF DESCRIPTION OF THE DRAWINGS

FIG. 1 is a block diagram illustrating an endoscope insertion shape observation apparatus according to a first embodiment;

FIG. 2 is a configuration diagram illustrating an entire configuration of a medical system including the endoscope insertion shape observation apparatus illustrated in FIG. 1;

FIG. 3 is an explanatory diagram for describing a method for using the endoscope insertion shape observation apparatus;

FIG. 4 is a block diagram illustrates an example of a specific configuration of a probe 21;

FIG. 5 is a flowchart for describing an operation according to the first embodiment;

FIG. 6A is an explanatory diagram illustrating an inserted state display image displayed on a display screen of a monitor 50;

FIG. 6B is an explanatory diagram illustrating an inserted state display image displayed on the display screen of the monitor 50;

FIG. 7 is a block diagram illustrating a second embodiment;

FIG. 8 is a flowchart for describing an operation according to the second embodiment;

FIG. 9A is an explanatory diagram illustrating an inserted state display image displayed on a display screen of a monitor 50;

FIG. 9B is an explanatory diagram illustrating an inserted state display image displayed on the display screen of the monitor 50;

FIG. 9C is an explanatory diagram illustrating an inserted state display image displayed on the display screen of the monitor 50;

FIG. 10 is a block diagram illustrating a third embodiment;

FIG. 11 is an explanatory diagram illustrating an example of a method for selecting a body outer shape image by a body outer shape image generation section 49;

FIG. 12A is an explanatory diagram illustrating an inserted state display image displayed on a display screen of a monitor 50;

FIG. 12B is an explanatory diagram illustrating an inserted state display image displayed on the display screen of the monitor 50;

FIG. 13 is a block diagram illustrating a fourth embodiment;

FIG. 14A is an explanatory diagram illustrating an inserted state display image displayed on a display screen of a monitor 50;

FIG. 14B is an explanatory diagram illustrating an inserted state display image displayed on the display screen of the monitor 50;

FIG. 15 is an explanatory diagram for describing a modification;

FIG. 16 is a block diagram illustrating a fifth embodiment;

FIG. 17A is an explanatory diagram illustrating an inserted state display image displayed on a display screen of a monitor 50;

FIG. 17B is an explanatory diagram illustrating an inserted state display image displayed on the display screen of the monitor 50;

FIG. 17C is an explanatory diagram illustrating an inserted state display image displayed on the display screen of the monitor 50;

FIG. 18 is a block diagram illustrating a sixth embodiment;

FIG. 19 is an explanatory diagram illustrating an example of a method for finding a dimension of a patient and a method for generating a body outer shape image;

FIG. 20 is an explanatory diagram illustrating an inserted state display image displayed on a display screen of a monitor 50;

FIG. 21 is a block diagram illustrating a seventh embodiment;

FIG. 22 is a block diagram illustrating an example in which a display problem of a loop portion can be solved;

FIG. 23 is an explanatory diagram illustrating an example of an insertion shape image displayed on a display screen of a monitor 50;

FIG. 24 is a block diagram illustrating another example in which a problem that the loop portion is difficult to confirm can be solved;

FIG. 25A is an explanatory diagram illustrating an example of an insertion shape image displayed on the display screen of the monitor 50;

FIG. 25B is an explanatory diagram illustrating an example of an insertion shape image displayed on the display screen of the monitor 50;

FIG. 26A is an explanatory diagram illustrating an example of a viewpoint display displayed on the display screen of the monitor 50;

FIG. 26B is an explanatory diagram illustrating an example of a viewpoint display displayed on the display screen of the monitor 50;

FIG. 26C is an explanatory diagram illustrating an example of a viewpoint display displayed on the display screen of the monitor 50; and

FIG. 27 is an explanatory diagram illustrating an inserted state display image subjected to multi-screen display on the display screen of the monitor 50.

### DETAILED DESCRIPTION

Exemplary embodiments will be described in detail below with reference to the drawings.

### First Exemplary Embodiment

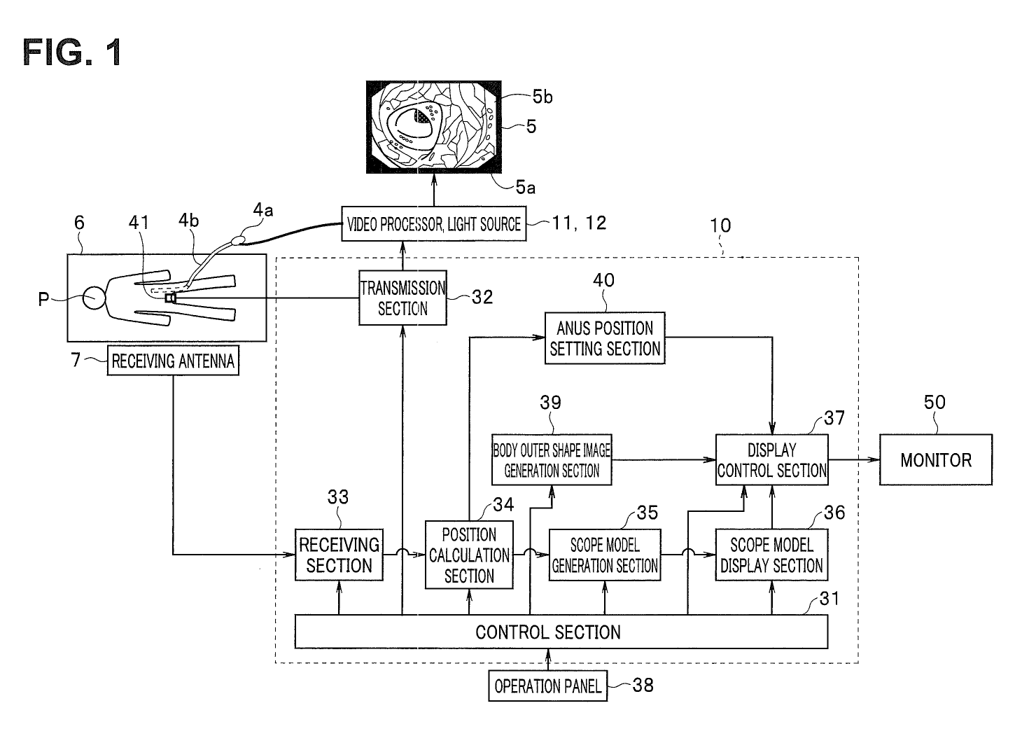

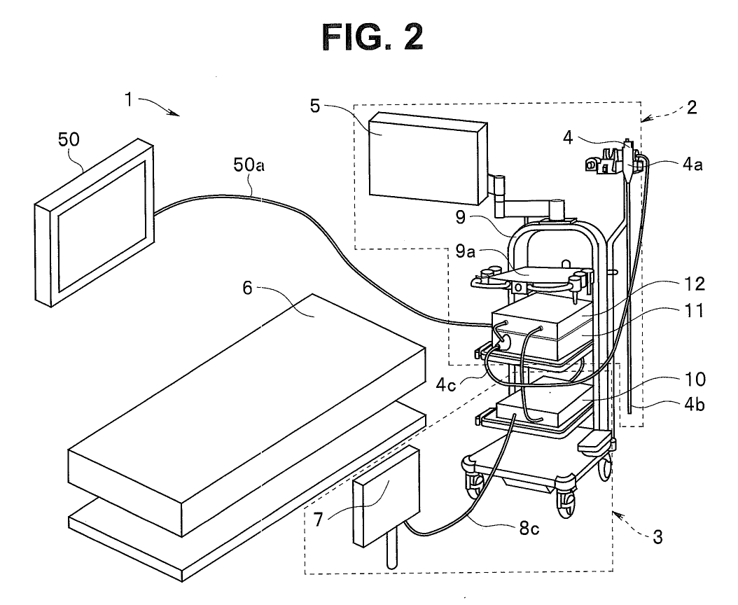

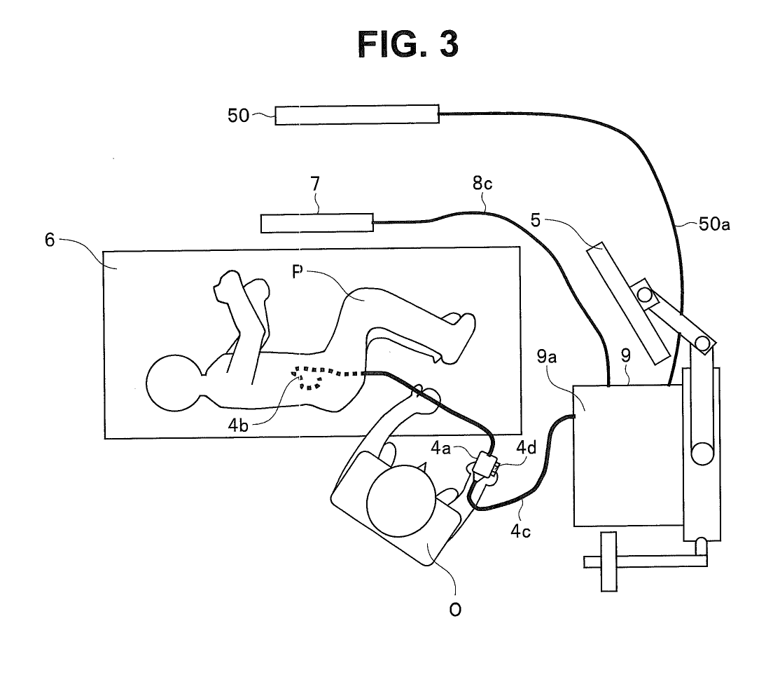

FIG. 1 is a block diagram illustrating an endoscope insertion shape observation apparatus according to a first exemplary embodiment. FIG. 2 is a configuration diagram illustrating an entire configuration of a medical system including the endoscope insertion shape observation apparatus illustrated in FIG. 1. FIG. 3 is an explanatory diagram for describing a method for using the endoscope insertion shape observation apparatus.

In the present exemplary embodiment, an insertion shape image representing an insertion shape of an endoscope is displayed while a body outer shape image representing an outer shape of a human body is displayed. In this case, in the present exemplary embodiment, the insertion shape image and the body outer shape image are aligned and displayed, to allow an operator to intuitively grasp an insertion position and shape in a body cavity.

In FIGS. 2 and 3, a medical system 1 is configured to include an endoscope apparatus 2 and an endoscope insertion shape observation apparatus 3. The endoscope apparatus 2 includes an endoscope 4, a light source device 11, a video processor 12, and a monitor 5. The endoscope 4 includes an insertion section 4b being elongated and having flexibility to be inserted into a body cavity of a subject P as an object to be inspected, an operation section 4a connected to a proximal end of the insertion section 4b and provided with various types of operation devices, and a cable 4c configured to connect the operation section 4a and the video processor 12.

FIG. 2 illustrates an example in which the light source device 11 and the video processor 12 are placed on a medical trolley 9. The monitor 5 is attached to a movable arm provided in the medical trolley 9. The endoscope 4 can be hung on a hook of the medical trolley 9.

FIG. 3 illustrates a state where the insertion section 4b is inserted into a large intestine from an anus of the subject P lying on a bed for inspection 6. FIG. 3 illustrates how an operator O grasps the operation section 4a and the insertion section 4b in the endoscope 4 connected to the video processor 12 on the medical trolley 9 via the cable 4c.

The light source device 11 generates illumination light for illuminating the subject. The illumination light from the light source device 11 is guided to a distal end portion of the insertion section 4b by a light guide inserted into the insertion section 4b in the endoscope 4, and is irradiated onto the subject from the distal end portion of the insertion section 4b. An image pickup device not illustrated is arranged in the distal end portion of the insertion section 4b, and reflected light (return light) from the subject, which has been reflected by the subject, is formed as an object optical image on a light receiving surface of the image pickup device. The image pickup device is controlled to be driven by the video processor 12, to convert the object optical image into an image signal and output the image signal to the video processor 12. The video processor 12 includes an image signal processing section not illustrated. The image signal processing section receives the image signal from the image pickup device to perform signal processing, and outputs an endoscope image after the signal processing to the monitor 5. Thus, an endoscope image 5b of the subject is displayed on a display screen 5a of the monitor 5, as illustrated in FIG. 1.

A bending portion is provided at a distal end of the insertion section 4b. The bending portion is driven to be bent by a bending knob 4d provided in the operation section 4a. The operator can push the insertion section 4b into the body cavity while bending the bending portion by operating the bending knob 4d.

In the present exemplary embodiment, the endoscope insertion shape observation apparatus 3 configured to observe an inserted state of the insertion section 4b includes a control unit 10, a probe for inserted state detection 21, a receiving antenna 7, and a monitor 50. Note that the monitor 50 is arranged at a position which can be observed by the operator O who inserts the insertion section 4b into the patient P, as illustrated in FIG. 3. The control unit 10 in the endoscope insertion shape observation apparatus 3 is placed on the medical trolley 9, and the probe for inserted state detection 21 is inserted into the insertion section 4b, as described below. The receiving antenna 7 is connected to the control unit 10 via a cable 8c.

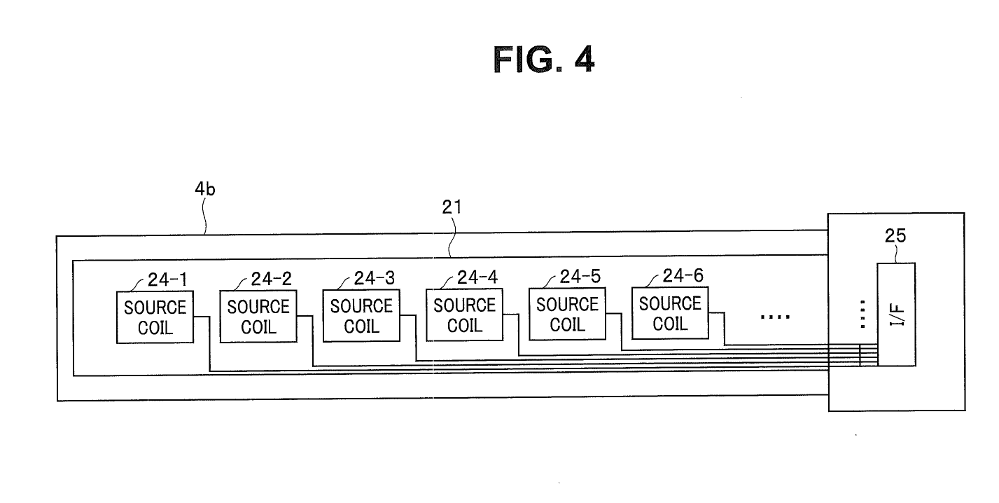

FIG. 4 is a block diagram illustrating an example of a specific configuration of the probe 21. As illustrated in FIG. 4, the probe 21 is inserted into a treatment instrument insertion channel not illustrated in the insertion section 4b. A plurality of transmission coils 24-1, 24-2, . . . (hereinafter merely referred to as transmission coils 24 when not required to be distinguished) are attached to the probe 21 with predetermined spacing, for example, along a probe axis of the probe 21. The probe 21 is inserted into the treatment instrument insertion channel to fix a proximal end or a rear end of the probe 21 so that the plurality of transmission coils 24-1, 24-2, . . . are arranged with predetermined spacing in an axial direction of the insertion section 4b.

Note that, although the probe 21 is inserted into and fixed to the treatment instrument insertion channel in the endoscope 4, to incorporate the transmission coils 24 into the insertion section 4b in the endoscope 4 in the present exemplary embodiment, the transmission coils 24 may be directly incorporated into the insertion section 4b in the endoscope 4.

The receiving antenna 7 includes a plurality of coil blocks not illustrated, and is arranged beside the bed 6, for example. Each of the coil blocks in the receiving antenna 7 includes three sense coils, for example, which are respectively wound in three directions such that their respective coil surfaces are perpendicular to one another. In the entire receiving antenna 7, four coil blocks, i.e., 12 sense coils, for example, are arranged. Each of the sense coils detects a signal proportional to an intensity of a magnetic field of an axial component perpendicular to the coil surface. For example, the coil block receives a generated magnetic field and converts the received magnetic field into a voltage signal, and outputs the voltage signal as a detection result. The control unit 10 controls respective operation states of the probe 21 and the receiving antenna 7.

The control unit 10 is provided with a control section 31, as illustrated in FIG. 1. The control section 31 can be configured by a processor using a CPU, for example, and may operate based on a program stored in a memory not illustrated. The control section 31 controls the entire control unit 10. Note that the memory not illustrated stores not only a program describing processing of the control section 31 but also data or the like used in position calculation, described below.

The control section 31 controls a transmission section 32. The transmission section 32 is configured by an FPGA, for example, and is controlled by the control section 31 to generate and output a sinusoidal signal, for example, for driving the probe 21. Note that the transmission section 32 can individually feed a sine wave to each of the coils 24 in the probe 21 upon being controlled by the control section 31. That is, the control section 31 can control to which of the transmission coils 24 in the probe 21 the sine wave is fed.

To each of the transmission coils 24, a high-frequency sine wave is fed from the control unit 10 via an I/F 25 (FIG. 4). Each of the transmission coils 24 radiates an electromagnetic wave with a magnetic field to surroundings when the high-frequency sine wave is applied thereto. Note that the control unit 10 can sequentially drive the transmission coils 24-1, 24-2, . . . at appropriate time intervals, e.g., at intervals of several milliseconds. The control unit 10 can also individually designate a timing at which each of the transmission coils 24-1, 24-2, . . . generates the magnetic field.

The receiving antenna 7 receives the magnetic field generated by the transmission coil 24 and converts the received magnetic field into a voltage signal using the sense coil. The receiving antenna 7 feeds the voltage signal to a receiving section 33 in the control unit 10 as a detection result. The receiving section 33 outputs, after the signal from the receiving antenna 7 has been fed and has been subjected to predetermined signal processing such as amplification processing, the signal to a position calculation section 34.

The position calculation section 34 includes a DSP, for example, and performs frequency extraction processing (Fourier transformation: FFT) for inputted digital data, separates the digital data into magnetic field detection information of frequency components respectively corresponding to the high-frequency sine waves applied to the transmission coils 24 and extracts the magnetic field detection information, and calculates respective spatial position coordinates of the transmission coils 24 provided in the probe 21 from respective digital data as the magnetic field detection information obtained by the separation. A calculation result of the position coordinates by the position calculation section 34 is fed to a scope model generation section 35. The scope model generation section 35 as an insertion shape image generation section connects the respective position coordinates of the transmission coils 24 and generates a linear image as an insertion shape image.

The insertion shape image generated by the scope model generation section 35 is fed to a scope model display section 36. The scope model display section 36 generates display data for displaying the insertion shape image generated by the scope model generation section 35 on the monitor 50, and outputs the generated display data to a display control section 37. The display control section 37 displays the insertion shape image on the display screen of the monitor 50 based on the inputted display data. The monitor 50 can be configured by an LCD, for example, and displays an insertion shape image based on a relative positional relationship between the transmission coil 24 and the receiving antenna 7 based on the display data.

The display data for the insertion shape image generated by the scope model display section 36 is generated using a coordinate system (hereinafter referred to as a measurement coordinate system) using a position of the antenna 7 as a reference. The display control section 37 performs coordinate transformation for displaying the insertion shape image at a predetermined position on the display screen of the monitor 50. That is, the display control section 37 performs coordinate transformation for transforming the measurement coordinate system into a display coordinate system for the inputted display data. The display control section 37 can display the insertion shape image in a predetermined orientation and size at the predetermined position on the display screen of the monitor 50 using the coordinate transformation. The display position, the orientation, and the size of the insertion shape image are changeable by an operator's operation.

An operation panel 38 can accept a user operation by the operator or the like and can output an operation signal based on the user operation to the control section 31. The operation panel 38 enables the operator to designate a change in the size of the insertion shape image, for example. The display control section 37 changes, when an instruction to change the size of the insertion shape image based on the user operation is issued from the control section 31, the size of the insertion shape image to be displayed on the monitor 50.

In the present exemplary embodiment, the control unit 10 is provided with a body outer shape image generation section 39 configured to output, when an operation by the operation panel 38 is detected, display data for a body outer shape image corresponding to a detected operation content. The body outer shape image is a human body chart or an anatomical chart, for example, capable of representing a body shape such as a shape itself of a body or a physical constitution of a patient. The body outer shape image may be an image which is schematic enough to find the body shape or may also be a detailed image including an image portion of organs such as an intestinal tract model of a large intestine. The body outer shape image is not limited to a 2D image. A technique for making stereoscopic viewing of a 3D image usable may be adopted.

The body outer shape image generation section 39 may hold the display data for the body outer shape image in a memory not illustrated and output the display data for the body outer shape image to the display control section 37 upon being controlled by the control section 31. The body outer shape image generation section 39 may also hold display data for a plurality of body outer shape images in the memory and output the display data for the one body outer shape image selected under the control of the control section 31 to the display control section 37.

For example, the body outer shape image generation section 39 may hold display data for a plurality of body outer shape images of sizes from a smallest S size to a largest XXL size in the memory, and the control section 31 may select the display data for the body outer shape image of the size based on a value of a BMI/a body height of the patient and output the selected display data to the display control section 37.

Further, the body outer shape image generation section 39 may be configured to generate the body outer shape image based on the body height and a waist dimension of the patient. The body outer shape image generation section 39 may also be configured to generate a body outer shape image including a navel and a diaphragm of a human body based on anatomical information.

In the present exemplary embodiment, to align and display the body outer shape image and the insertion shape image, the body outer shape image and the insertion shape image are simultaneously displayed with a predetermined position of the body outer shape image (hereinafter referred to as a body outer shape image reference position) corresponding to a predetermined position of the subject (hereinafter referred to as a subject reference position) and a predetermined position of the insertion shape image (hereinafter referred to as an insertion shape image reference position) corresponding to the subject reference position matched with each other. For example, a position of the anus of the subject P is set from the spatial position coordinates calculated by the position calculation section 34 as the subject reference position.

To set the subject reference position of the subject P, a marker 41 is adopted, for example. The marker 41 contains a transmission coil not illustrated, and a high-frequency sine wave is applied to the transmission coil from the transmission section 32. The marker 41 generates a magnetic field when the high-frequency sine wave is applied thereto from the transmission section 32. The magnetic field is received by the receiving antenna 7, and a detection result by the receiving antenna 7 is fed to the position calculation section 34 via the receiving section 33. As a result, the position calculation section 34 can acquire position coordinates of the marker 41 in the measurement coordinate system.

The operator can obtain position coordinates of the anus position from the position calculation section 34 when the control section 31 controls the transmission section 32 to output the high-frequency sine wave to the marker 41 with the marker 41 arranged in the vicinity of the anus of the subject P. The position coordinates are fed to an anus position setting section 40. The anus position setting section 40 holds the position coordinates of the anus position of the subject P while outputting the held position coordinates to the display control section 37.

Note that, when the marker 41 is affixed to the vicinity of the anus of the subject P, the control section 31 controls the transmission section 32 to output the high-frequency sine wave to the marker 41 at a predetermined timing so that position coordinates of the anus position (hereinafter referred to as anus position coordinates) of the subject P at the timing are held in the anus position setting section 40. As a result, even if the anus position of the subject P changes, information about the actual anus position is fed to the display control section 37.

The display control section 37 displays the body outer shape image on the display screen of the monitor 50 with the body outer shape image reference position matched with a predetermined position on the display screen (hereinafter referred to as a display reference position). For example, the display control section 37 sets the display reference position to a lowest end at a center in a horizontal direction of the display screen, and displays the body outer shape image such that the anus position of the body outer shape image (the body outer shape image reference position) is positioned at the display reference position. The display control section 37 also displays the insertion shape image such that an image portion corresponding to the anus position of the insertion shape image is positioned at the lowest end at the center in the horizontal direction of the display screen as the display reference position.

Note that, although the anus position of the subject P is found using the marker 41 in the above description, the anus position may be found using the probe 21 inserted into the insertion section 4b. For example, the control section 31 may apply the high-frequency sine wave from the transmission section 32 to the transmission coil at the distal end of the probe 21 by an operation of the operation panel 38 or the like with the transmission coil at the distal end of the probe 21 having reached the anus position of the subject P. A magnetic field by the coil is received by the receiving antenna 7, and a detection result by the receiving antenna 7 is fed to the position calculation section 34 via the receiving section 33. As a result, the position calculation section 34 can acquire a coil position at the distal end of the probe in the measurement coordinate system, i.e., the anus position coordinates.

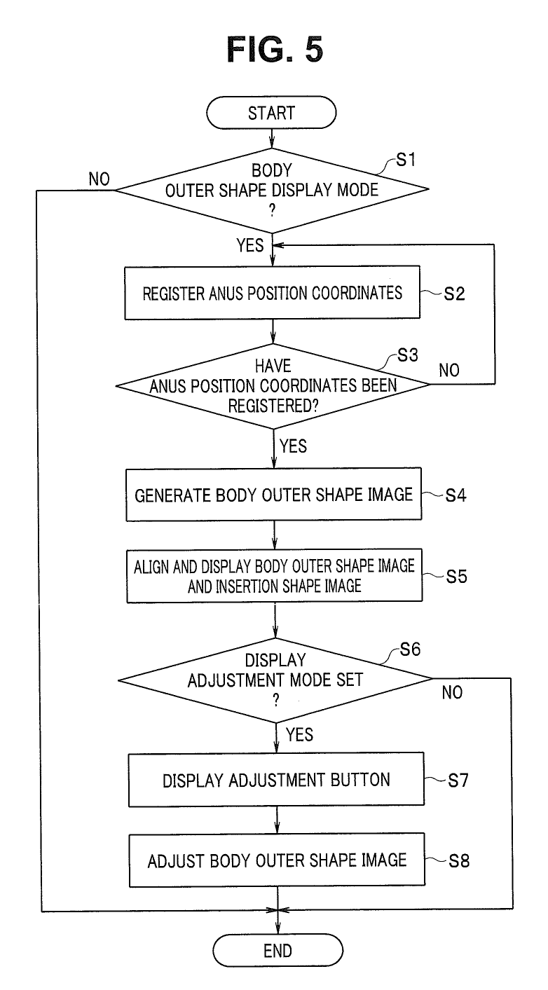

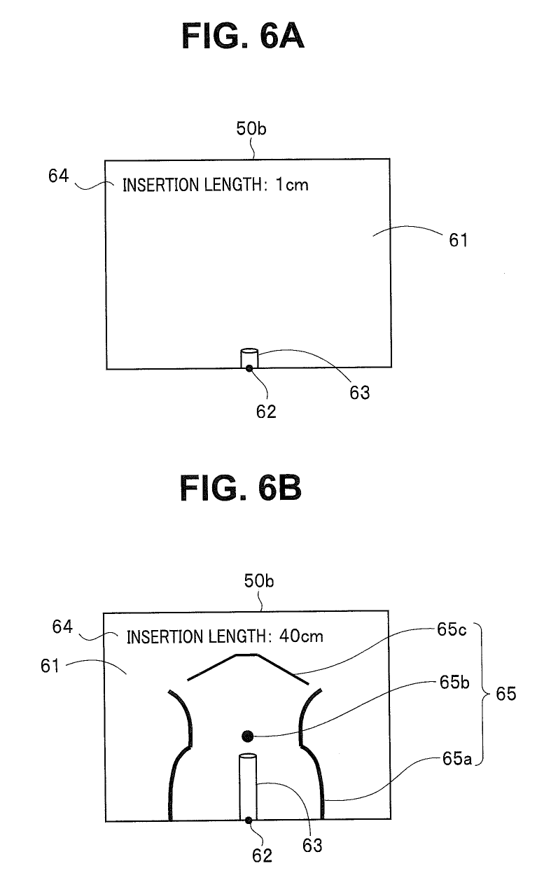

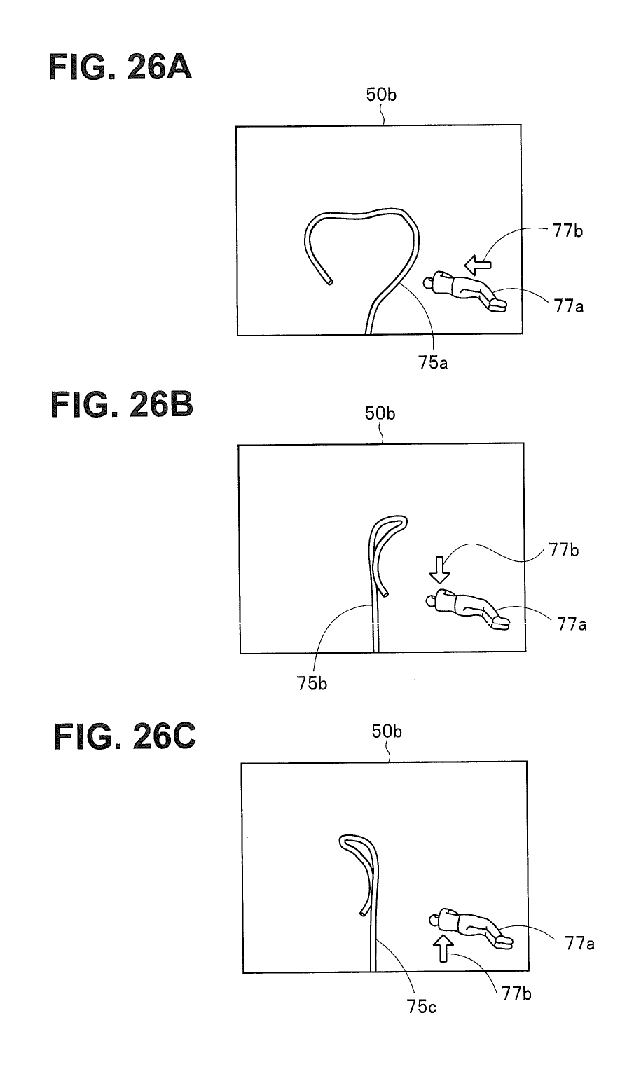

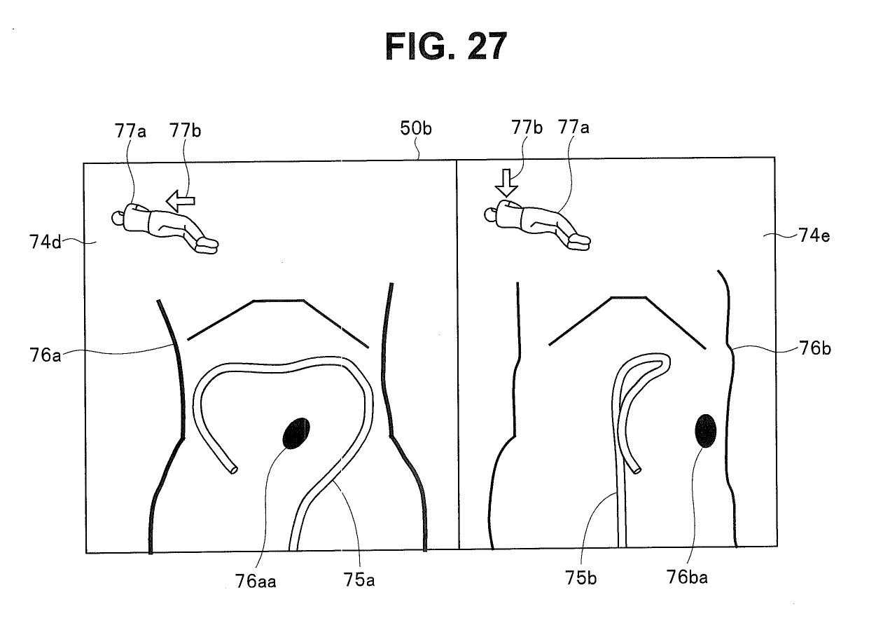

Then, an operation according to the exemplary embodiment thus configured will be described with reference to FIGS. 5, 6A, 6B, 26A to 26C, and 27. FIG. 5 is a flowchart for describing an operation according to the first exemplary embodiment. FIGS. 6A and 6B are explanatory diagrams each illustrating an inserted state display image displayed on the display screen of the monitor 50. FIGS. 26A to 26C are explanatory diagrams each illustrating a viewpoint display displayed on the display screen of the monitor 50. FIG. 27 is an explanatory diagram illustrating an inserted state display image subjected to multi-screen display on the display screen of the monitor 50.

As illustrated in FIG. 3, it is assumed that the operator inserts the insertion section 4b into the large intestine from the anus of the subject P lying in a lateral position on the bed for inspection 6. The endoscope insertion shape observation apparatus 3 finds three-dimensional position coordinates of each of the plurality of transmission coils 24 in the probe 21 contained in the insertion section 4b at predetermined time intervals. That is, the control section 31 in the control unit 10 controls the transmission section 32 to feed a high-frequency signal at a predetermined timing to each of the transmission coils 24-1, 24-2, . . . in the probe 21. The transmission coils 24-1, 24-2, . . . to which the high-frequency signal has been fed generate an electromagnetic wave with a magnetic field. The magnetic field is received in each of the coil blocks in the receiving antenna 7, and a detection result corresponding to an intensity of the magnetic field is accepted in the position calculation section 34 via the receiving section 33 in the control unit 10.

The position calculation section 34 is given information about a driving timing of each of the transmission coils 24-1, 24-2, . . . from the control section 31, and finds, for each of the transmission coils 24-1, 24-2, . . . , three-dimensional position coordinates of the transmission coil according to a known position estimation algorithm from the detection result outputted by the coil block.

The position coordinates are fed to the scope model generation section 35. The scope model generation section 35 generates an insertion shape image based on the position coordinates. The probe 21 is inserted into the treatment instrument insertion channel in the insertion section 4b, and the transmission coils 24 are respectively arranged at known positions with predetermined spacing along a shape of the insertion section 4b. That is, the positions of the transmission coils 24 respectively represent discrete positions in the insertion section 4b. The scope model generation section 35 interpolates the discrete position, to generate an insertion shape image corresponding to the schematic shape of the insertion section 4b. Note that the insertion shape image is found in the measurement coordinate system.

The scope model generation section 35 gives the generated insertion shape image to the scope model display section 36. The scope model display section 36 generates display data based on the insertion shape image, and outputs the generated display data to the display control section 37. The display control section 37 displays the insertion shape image on a display screen 50b of the monitor 50. FIG. 6A illustrates an inserted state display image 61 displayed on the display screen 50b in this case. An insertion shape image 63 is displayed in the inserted state display image 61. Note that the inserted state display image 61 illustrated in FIG. 6A represents an example in which the insertion shape image 63 is displayed using a reference position 62, described below, as a reference.

In the present exemplary embodiment, not only the insertion shape image but also the body outer shape image can be displayed on the monitor 50. The control section 31 judges whether or not a body outer shape display mode is set in step S1 illustrated in FIG. 5. If the body outer shape display mode is not set, the processing ends. If the body outer shape display mode is set, then in step S2, the control section 31 registers anus position coordinates.

That is, the control section 31 controls the transmission section 32, to apply a high-frequency sine wave to the marker 41. As a result, the marker 41 generates an electromagnetic wave with a magnetic field, and the magnetic field is received in each of the coil blocks in the receiving antenna 7. Accordingly, a detection result corresponding to an intensity of the magnetic field is accepted in the position calculation section 34 from the receiving antenna 7 via the receiving section 33 in the control unit 10.

The position calculation section 34 acquires anus position coordinates in the measurement coordinate system of the marker 41 according to a known position estimation algorithm from the detection result based on the magnetic field generated by the marker 41. The anus position coordinates are given to and held in the anus position setting section 40.

The control section 31 judges in step S3 whether or not the anus position coordinates have been registered, and controls each of the sections to perform work for registering the anus position coordinates in step S2 until the anus position coordinates are registered. If the anus position coordinates are registered, the processing proceeds to step S4.

The control section 31 causes the operation panel 38 to control the body outer shape image generation section 39 to generate a body outer shape image in step S4. Display data for the body outer shape image is fed to the display control section 37. To the display control section 37, the anus position coordinates and the insertion shape image 63 are also respectively given from the anus position setting section 40 and the scope model display section 36. The display control section 37 is controlled by the control section 31, to display the insertion shape image 63 and a body outer shape image 65 such that the anus position coordinates are positioned at the display reference position 62 at a lowest end at a center in a horizontal direction on the display screen 50b of the monitor 50, for example. That is, the display control section 37 displays the insertion shape image 63 to match a portion of the anus position coordinates in the measurement coordinate system among portions of the insertion shape image 63 with the reference position 62. The display control section 37 also displays the body outer shape image 65 to match an image portion corresponding to an anus of the body outer shape image 65 with the reference position 62 (step S5).

FIG. 6B illustrates the inserted state display image 61 displayed on the display screen 50b in this case. In the inserted state display image 61 illustrated in FIG. 6B, the insertion shape image 63 and the body outer shape image 65 are synthesized while being aligned at the reference position 62. Note that the body outer shape image 65 includes a line image 65a representing a contour of a human body, an image 65b representing a navel portion of the human body, and an image 65c representing a diaphragm.

Note that, although an example in which the reference position 62 is set at the lowest end at the center in the horizontal direction on the display screen 50b is illustrated in FIGS. 6A and 6B, the present exemplary embodiment is not limited to this. The reference position 62 can be set to an appropriate screen position.

In the present exemplary embodiment, the insertion shape image 63 and the body outer shape image 65 can be synthesized and displayed in a relatively small calculation processing amount because a portion of the insertion shape image 63 positioned at the anus position and an anus position in the body outer shape image 65 are only matched with the reference position 62 on the display screen 50b. However, in the body outer shape image generation section 39, it is considered that the body outer shape image 65 has not been generated by measurement for an actual subject P, and a size of the body outer shape image 65 does not necessarily correspond to the insertion shape image 63.

Therefore, the control section 31 can perform adjustment of top, bottom, left, and right display positions, scaling, inclination, and viewpoint switching of the body outer shape image 65 by an operator's operation, and judges in step S6 whether or not the operator has designated a display adjustment mode. Note that a viewpoint indicates from which direction the insertion shape image 63 which is being displayed is viewed to represent an insertion shape of an endoscope. When the display adjustment mode is designated, the control section 31 displays an operation button (adjustment button) for the adjustment on an LCD screen not illustrated of the operation panel 38, for example (step S7).

For example, the display control section 37 displays on the display screen 50b of the monitor 50 the insertion shape image 63 from the scope model display section 36 and a viewpoint display including a viewpoint position display 77b representing a viewpoint position of a current display and a body marker 77a which indicates a body position of a patient at a glance, as illustrated in FIGS. 26A to 26C. FIG. 26A indicates that a viewpoint is on a front surface of the subject P by the body marker 77a and the viewpoint position display 77b, FIG. 26B indicates that a viewpoint is on a right side surface of the subject P by the body marker 77a and the viewpoint position display 77b, and FIG. 26C indicates that a viewpoint is on a left side surface of the subject P by the body marker 77a and the viewpoint position display 77b. When the viewpoint display is switched using the operation button (adjustment button) on the operation panel 38, the insertion shape image 63 viewed from each of the viewpoint positions is displayed. Note that, although an example in which the body outer shape image 65 is not displayed is illustrated in FIGS. 26A to 26C, the body outer shape image 65 may be displayed in the viewpoint display.

FIG. 27 illustrates an example in which an inserted state display image including a viewpoint display is subjected to multi-screen display. The display control section 37 can not only switch and display inserted state display images respectively viewed from different viewpoints but also simultaneously display the inserted state display images viewed from the different viewpoints, and can also further display a viewpoint display in each of the inserted state display images. FIG. 27 illustrates an example in which an inserted state display image 74d viewed from the side of an abdomen of the patient P is displayed on a left side of the display screen 50b of the monitor 50 and an inserted state display image 74e viewed from the side of a right body side of the patient P is displayed on a right side of the display screen 50b. The inserted state display image 74d includes a body outer shape image 76a including an image portion of a navel portion 76aa and an insertion shape image 75a. The inserted state display image 74d also includes a body marker 77a and a viewpoint position display 77b each indicating that a viewpoint is on the side of the abdomen of the patient P on an upper side of the screen. The inserted state display image 74e includes a body outer shape image 76b including an image portion of a navel portion 76ba and an insertion shape image 75b. The inserted state display image 74e also includes a body marker 77a and a viewpoint position display 77b each indicating that a viewpoint is on the side of a right body side of the patient P on an upper side of the screen.

Note that, although FIG. 27 illustrates an example in which two inserted state display images which differ in viewpoints are subjected to dual-screen display, three or more inserted state display images which differ in viewpoints can also be subjected to multi-screen display.

The operator can perform adjustment of top, bottom, left, and right display positions, scaling, inclination, viewpoint switching of the body outer shape image 65 using the operation panel 38. The display control section 37 is controlled according to an operator's operation, to perform adjustment of top, bottom, left, and right display positions, scaling, inclination, and viewpoint switching of the body outer shape image 65 (step S8). As a result, the operator or the like can more easily grasp into which position the insertion section 4b has been inserted.

Accordingly, in the present exemplary embodiment, the insertion shape image of the endoscope insertion section and the body outer shape image representing the body shape of the subject are aligned, synthesized, and displayed. The insertion shape image and the body outer shape image make it possible to easily grasp at which position of the body outer shape the insertion section is positioned. That is, it can be relatively easily guessed at which position in the body cavity of the subject the insertion section is positioned. As a result, the operator and a medical advisor, for example, can easily grasp the inserted state.

### Second Exemplary Embodiment

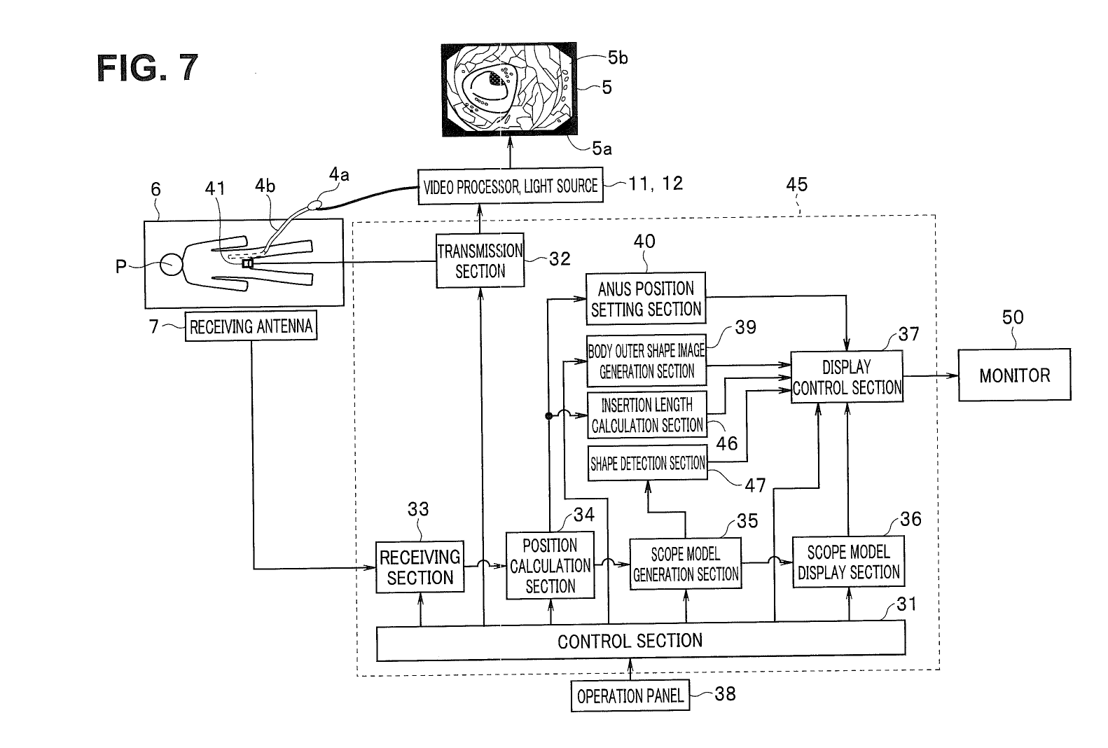

FIG. 7 is a block diagram illustrating a second exemplary embodiment. In FIG. 7, similar components to the components illustrated in FIG. 1 are assigned the same reference numerals, and description of the components is omitted. In the first exemplary embodiment, the example in which display or non-display of the body outer shape image is switched by an operator's operation is illustrated. The present exemplary embodiment illustrates an example in which display or non-display of a body outer shape image is switched depending on an insertion length and an insertion shape.

A control unit 45 in the present exemplary embodiment differs from the control unit 10 illustrated in FIG. 1 in that an insertion length calculation section 46 and a shape detection section 47 are added. The insertion length calculation section 46 calculates a length of an insertion section 4b inserted into a body cavity. A portion of the insertion section 4b in which a transmission coil 24, position coordinates detected in a position calculation section 34 of which correspond to anus position coordinates, among transmission coils 24 is arranged is positioned in an anus, and a portion from the position of the coil 24 to a distal end of the insertion section 4b is inserted into the body cavity. A position from the distal end of the insertion section 4b of each of the transmission coils 24 inserted into the insertion section 4b is known. The insertion length calculation section 46 calculates a length from the position of the coil 24 positioned in the anus to the distal end of the insertion section 4b as an insertion length. The insertion length calculation section 46 outputs information about the calculated insertion length to a display control section 37.

In the present exemplary embodiment, the display control section 37 is controlled by a control section 31, to display a body outer shape image when the calculated insertion length is in a predetermined length range. In inspection of a large intestine, for example, an operator may confirm an inserted state of the insertion section 4b in a bending portion such as a sigmoid colon. A distance of a position of the sigmoid colon from the anus is schematically known. The display control section 37 may display a body outer shape image when a distal end portion of the insertion section 4b is positioned in the vicinity of a sigmoid colon portion, for example.

The shape detection section 47 can detect a predetermined shape in the body cavity of the insertion section 4b based on an insertion shape image from a scope model generation section 35. For example, the shape detection section 47 can detect which of a linear shape, a stick shape, a loop shape, and the like is a shape of the insertion section 4b using a known method. The shape detection section 47 outputs information about the detected shape to the display control section 37.

In the present exemplary embodiment, the display control section 37 displays the body outer shape image when the detected shape is a predetermined shape. For example, the operator may confirm the inserted state when the shape of the insertion section 4b in the body cavity, i.e., the insertion shape image is a loop shape or a stick shape. Therefore, the display control section 37 may cause a shape pattern representing a loop shape or a stick shape to be stored in the shape detection section 47, for example, and when it is detected that the insertion shape image has formed the shape pattern, the display control section 37 may cause the body outer shape image to be displayed.

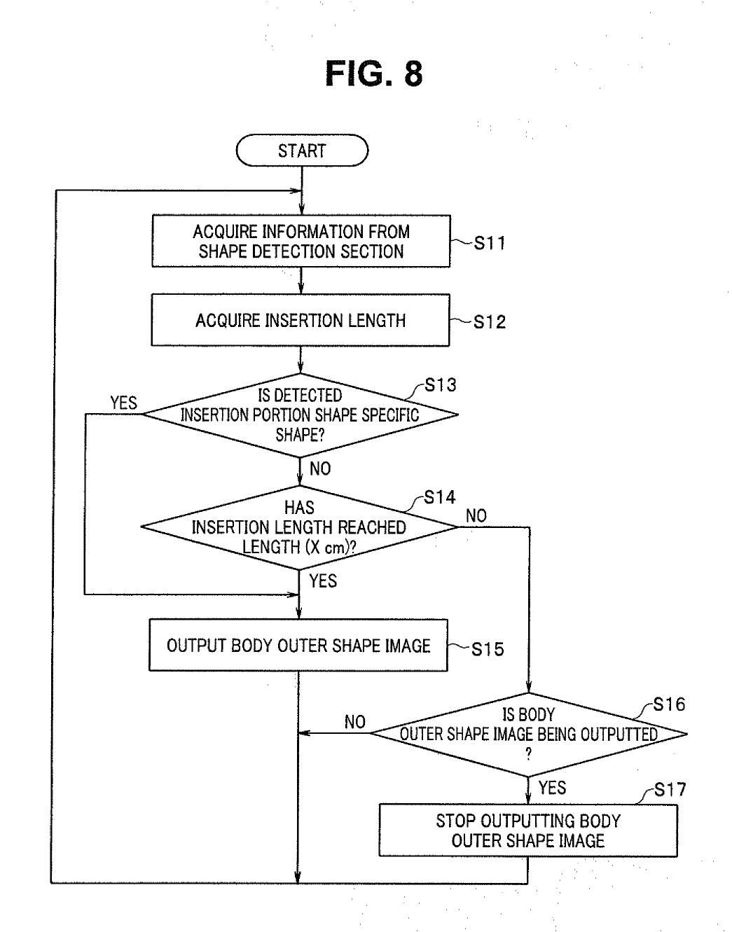

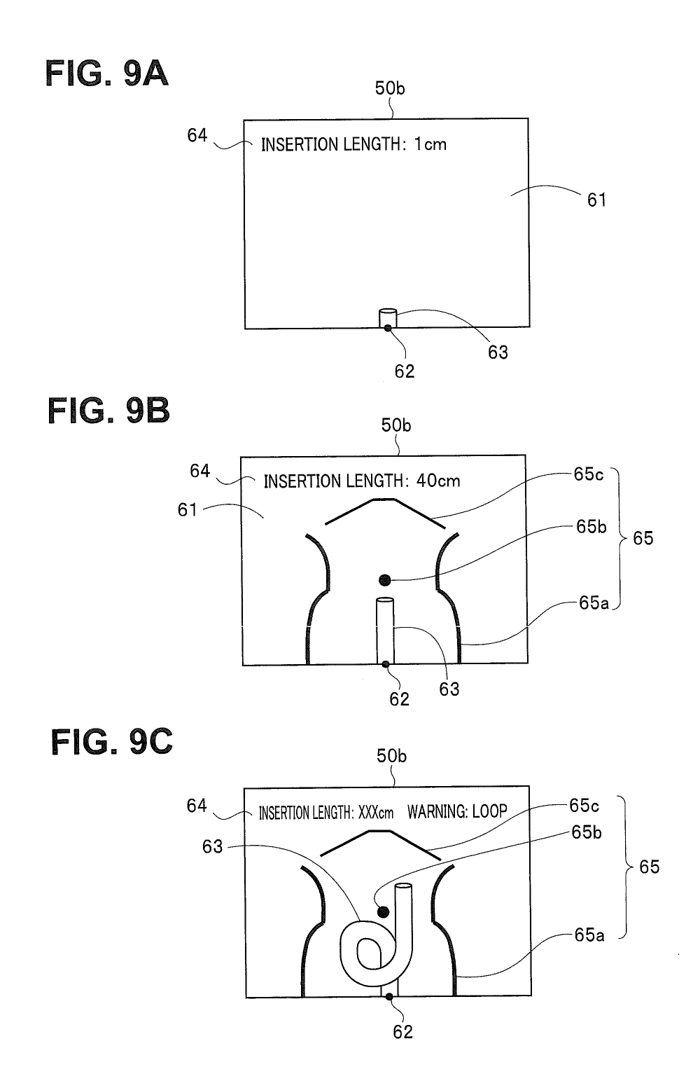

Then, an operation according to the exemplary embodiment thus configured will be described with reference to FIGS. 8 and 9. FIG. 8 is a flowchart for describing an operation according to a second exemplary embodiment. FIGS. 9A to 9C are explanatory diagrams each illustrating an inserted state display image displayed on a display screen of a monitor 50.

In step S11 illustrated in FIG. 8, the display control section 37 acquires information about an insertion shape from the shape detection section 47. The display control section 37 also acquires information about an insertion length from the insertion length calculation section 46 (step S12). The display control section 37 judges in step S13 whether or not the detected insertion shape is a specific shape such as a loop shape. The display control section 37 also judges in step S14 whether or not the insertion length has reached a length (e.g., 40 cm) of a specific site.

If both respective judgment results in steps S13 and S14 are negative, then in step S16, the display control section 37 judges whether or not a body outer shape image is being outputted (displayed). If the body outer shape image is being displayed, then in step S17, the display control section 37 stops outputting (does not display) the body outer shape image, and the processing returns to step S11. Note that, if the body outer shape image is not being displayed, the display control section 37 returns the process from step S16 to step S11. That is, if both the respective judgment results in steps S13 and S14 are negative, the body outer shape image is not displayed.

If the insertion length is 1 cm, and the insertion shape is not the specific shape (e.g., the loop shape), for example, both the respective judgment results in steps S13 and S14 are negative, and an inserted state display image 61 illustrated in FIG. 9A is displayed. In an example illustrated in FIG. 9A, an insertion length display 64 indicating that an insertion length is 1 cm and an insertion shape image 63 having a substantially linear shape are displayed in the inserted state display image 61.

If the judgment result in either one of steps S13 and S14 is positive, the processing proceeds to step S15. In step S15, the display control section 37 outputs (displays) the body outer shape image, and the processing returns to step S11.

If the insertion shape is a linear shape, and the insertion length is 40 cm, for example, the processing proceeds from step S14 to step S15. In step S15, an inserted state display image 61 illustrated in FIG. 9B is displayed. In an example illustrated in FIG. 9B, an insertion length display 64 indicating that the insertion length is 40 cm and an insertion shape image 63 having a substantially linear shape are displayed in the inserted state display image 61. Further, a body outer shape image 65 is displayed in the inserted state display image 61. The body outer shape image 65 includes a line image 65a representing a contour of a human body, an image 65b representing a navel portion of the human body, and an image 65c representing a diaphragm.

If the insertion length is XXX cm exceeding 40 cm, and the insertion shape is a loop shape, for example, the processing proceeds from step S13 to step S15. In step S15, an inserted state display image 61 illustrated in FIG. 9C is displayed. In an example illustrated in FIG. 9C, an insertion length display 64 indicating that the insertion length is XXX cm and an insertion shape image 63 having a loop shape are displayed in the inserted state display image 61. Further, a similar body outer shape image 65 to the body outer shape image illustrated in FIG. 9B is displayed in the inserted state display image 61.

Accordingly, in the present exemplary embodiment, a similar effect to the effect in the first exemplary embodiment is obtained while display or non-display of the body outer shape image can be switched depending on a result of at least one of the insertion length and the insertion shape. As a result, in a scene in which the insertion shape is securely confirmed, e.g., a case where the insertion section is to be inserted into a site in a bent state such as a sigmoid colon and a case where the insertion section has a loop shape, for example, the body outer shape image can be displayed, and the inserted state can be easily confirmed.

### Third Exemplary Embodiment

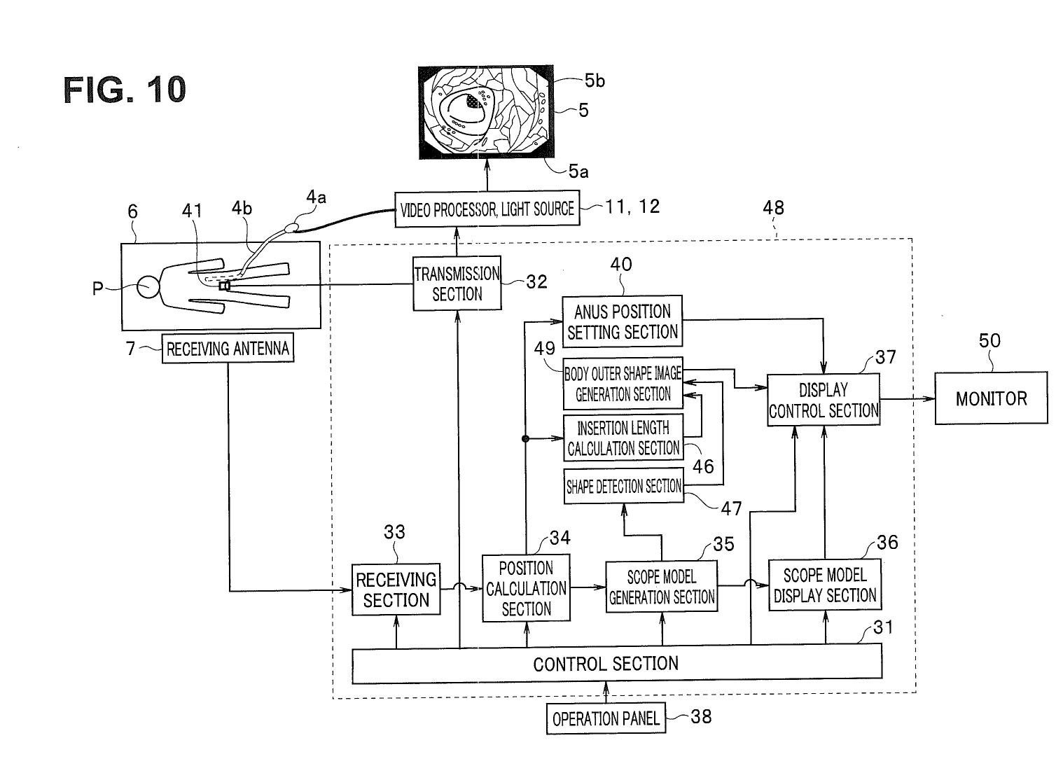

FIG. 10 is a block diagram illustrating a third exemplary embodiment. In FIG. 10, similar components to the components illustrated in FIG. 7 are assigned the same reference numerals, and description of the components is omitted. Although the example in which display or non-display of the body outer shape image is switched depending on the insertion length and the insertion shape has been described in the second exemplary embodiment, the present exemplary embodiment illustrates an example in which a type of a body outer shape image is switched depending on an insertion length and an insertion shape.

A control unit 48 in the present exemplary embodiment differs from the control unit 45 illustrated in FIG. 7 in that a body outer shape image generation section 49 is adopted instead of the body outer shape image generation section 39. The body outer shape image generation section 49 holds respective display data for a plurality of types of body outer shape images in a memory not illustrated, and selects one of the body outer shape images and outputs the selected body outer shape image to a display control section 37 according to at least one of a calculation result by an insertion length calculation section 46 and a detection result by a shape detection section 47. Note that the body outer shape image generation section 49 may stop outputting the body outer shape image to the display control section 37 depending on at least one of the calculation result by the insertion length calculation section 46 and the detection result by the shape detection section 47.

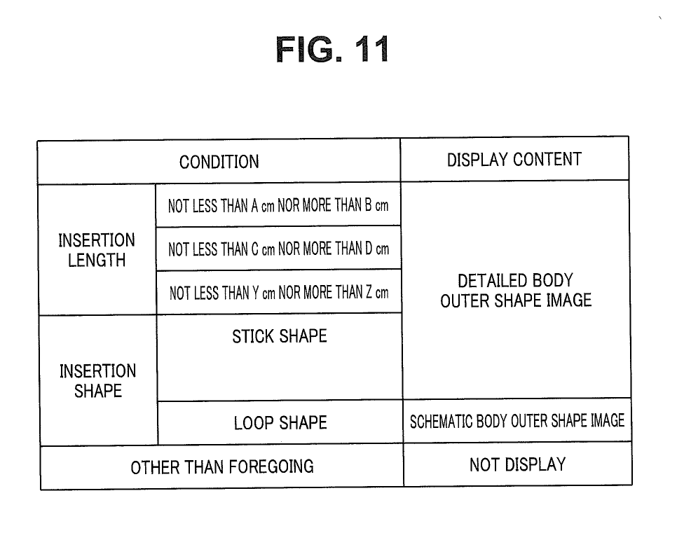

FIG. 11 is an explanatory diagram illustrating an example of a method for selecting the body outer shape images by the body outer shape image generation section 49. An example illustrated in FIG. 11 indicates that the body outer shape image generation section 49 selects the detailed body outer shape image and outputs display data for the selected body outer shape image to the display control section 37 when the insertion length is not less than A cm nor more than B cm, not less than C cm nor more D cm, or not less than Y cm nor more than Z cm. Note that dimensions of the insertion length are set to a length including a site, into which insertion is not easily performed, such as a position of a sigmoid colon, and a site an insertion progress of which is desired to be grasped, for example. For example, a position of the sigmoid colon is in a range of 15 cm to 30 cm from an anus. Accordingly, a detection range of the insertion length is set to not less than 15 cm nor more than 30 cm. The example illustrated in FIG. 11 also indicates that the body outer shape image generation section 49 selects the detailed body outer shape image and outputs display data for the selected body outer shape image to the display control section 37 when the insertion shape is a stick shape, and selects the schematic body outer shape image and outputs display data for the selected body outer shape image to the display control section 37 when the insertion shape is a loop shape. The example illustrated in FIG. 11 also indicates that the body outer shape image generation section 49 does not output the body outer shape image under conditions other than the conditions.

Note that the detailed body outer shape image is an image in which a contour shape of a body shape and a state of internal organs are respectively close to actual shapes, for example, and the schematic body outer shape image is an image schematically representing a contour of the body shape, for example.

The selection method illustrated in FIG. 11 may be set in the body outer shape image generation section 49 based on information stored in the memory not illustrated by a control section 31, for example.

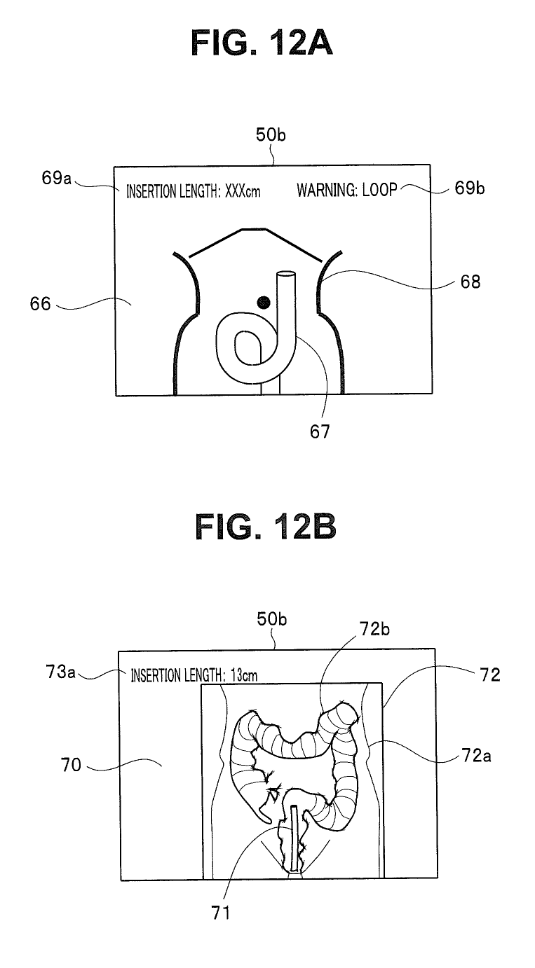

Then, an operation according to the exemplary embodiment thus configured will be described with reference to explanatory diagrams of FIGS. 12A and 12B.

FIGS. 12A and 12B are explanatory diagrams each illustrating an inserted state display image displayed on a display screen of a monitor 50.

The body outer shape image generation section 49 acquires information about an insertion shape from the shape detection section 47. The body outer shape image generation section 49 also acquires information about an insertion length from the insertion length calculation section 46. The body outer shape image generation section 49 is controlled by the control section 31, to select the body outer shape images depending on the insertion length while selecting the body outer shape image corresponding to the insertion shape.

For example, it is assumed that the insertion length is none of not less than A cm nor more than B cm, not less than C cm nor more D cm, and not less than Y cm nor more than Z cm, and the insertion shape is neither a stick shape nor a loop shape. In this case, the body outer shape image generation section 49 does not output display data for the body outer shape image. That is, in this case, the display control section 37 displays an inserted state display image in which only an insertion shape image is displayed.

For example, it is assumed that the insertion length is none of not less than A cm nor more than B cm, not less than C cm nor more than D cm, and not less than Y cm nor more than Z cm, and the insertion shape is a loop shape. In this case, the body outer shape image generation section 49 selects a schematic body outer shape image and outputs display data for the selected body outer shape image to the display control section 37. In this case, the display control section 37 displays an inserted state display image obtained by synthesizing the insertion shape image and the schematic body outer shape image. FIG. 12A illustrates an inserted state display image 66 displayed on a display screen 50b in this case.

The inserted state display image 66 includes an insertion shape image 67 having a loop shape and a schematic body outer shape image 68. The inserted state display image 66 also includes a warning display 69b indicating that the insertion shape is a loop shape in addition to an insertion length display 69b indicating that the insertion length is XXX cm.

When the inserted state display image 66 is referred to, an operator can easily grasp a warning that the insertion shape of an insertion section 4b is a loop shape and an approximate position in a body cavity of the insertion section 4b. In this case, a detailed insertion position of the insertion section 4b may not often be required to be known, and the loop shape is more easily intuitively grasped by displaying the schematic body outer shape image 68 than displaying a detailed body outer shape image.

For example, it is assumed that the insertion length is not less than A cm nor more than B cm, not less than C cm nor more than D cm, or not less than Y cm nor more than Z cm, or the insertion shape is a stick shape. In this case, the body outer shape image generation section 49 selects a detailed body outer shape image and outputs display data for the selected body outer shape image to the display control section 37. In this case, the display control section 37 displays an inserted state display image obtained by synthesizing the insertion shape image and the detailed body outer shape image. FIG. 12B illustrates an inserted state display image 70 displayed on the display screen 50b in this case.

The inserted state display image 70 includes an insertion shape image 71 and a detailed body outer shape image 72. The inserted state display image 70 also includes an insertion length display 73a indicating that an insertion length is 13 cm included in not less than A cm nor more than B cm, for example.

The detailed body outer shape image 72 includes a body outer shape contour image 72a having a shape close to an actual body shape of a human body while including an intestinal tract model image 72b. The insertion shape image 71 indicates into which position in the intestinal tract model image 72b the insertion section 4b has been inserted.

When the inserted state display image 70 is referred to, an operator can easily grasp at which position in a body cavity the insertion section 4b is positioned. For example, the detailed body outer shape image 72 is displayed when the insertion section 4b has reached the vicinity of the sigmoid colon. The detailed body outer shape image 72 is displayed at such a site into which the insertion section 4b is not easily inserted. Therefore, the operator can reliably grasp into which position in an intestinal tract the insertion section 4b has been inserted, which is significantly effective for work for inserting the insertion section 4b.

Accordingly, in the present exemplary embodiment, a similar effect to the effect in the first exemplary embodiment is obtained while the body outer shape images of different types can be displayed depending on a result of at least one of the insertion length and the insertion shape so that an appropriate body outer shape image corresponding to each scene to be inserted can be displayed. As a result, the operator can easily and reliably confirm an inserted state.

### Fourth Exemplary Embodiment

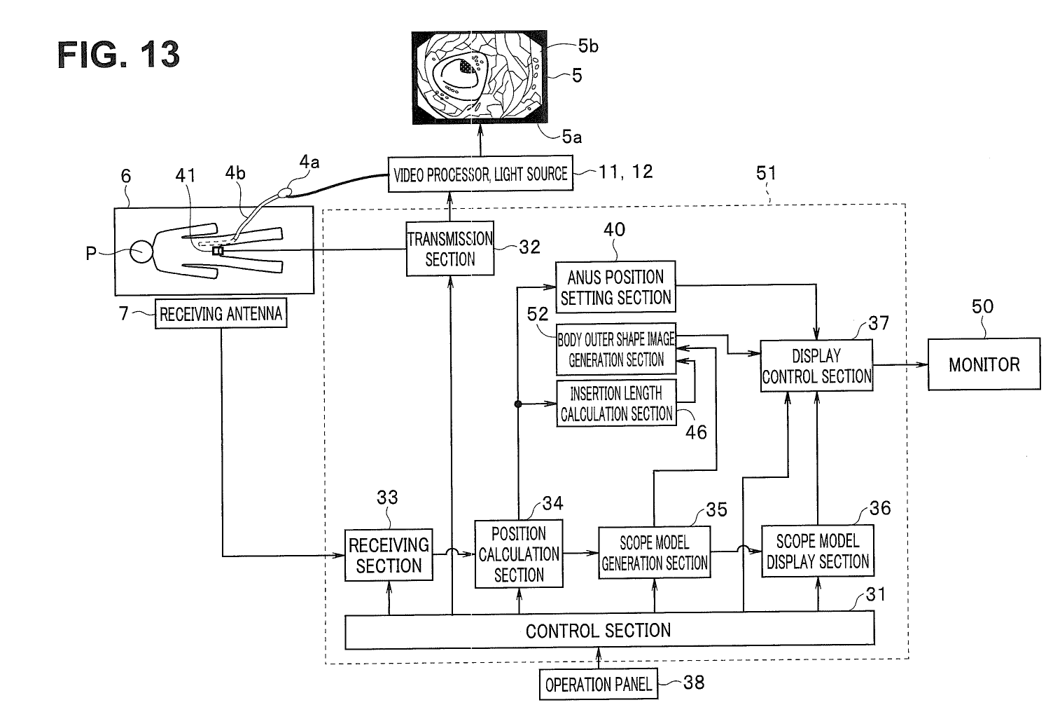

FIG. 13 is a block diagram illustrating a fourth exemplary embodiment. In FIG. 13, similar components to the components illustrated in FIG. 10 are assigned the same reference numerals, and description of the components is omitted. In the third exemplary embodiment, one of the plurality of types of body outer shape images is selected and displayed according to a result of at least one of the insertion length and the insertion shape. On the other hand, in the present exemplary embodiment, a body outer shape image is deformed and displayed according to an insertion length and an insertion shape image.

A control unit 51 in the present exemplary embodiment differs from the control unit 48 illustrated in FIG. 10 in that a body outer shape image generation section 52 is adopted instead of the body outer shape image generation section 49. The body outer shape image generation section 52 holds display data for a detailed body outer shape image including an intestinal tract model in a memory not illustrated. The body outer shape image generation section 52 also deforms an intestinal tract model image in the body outer shape image and outputs the deformed intestinal tract model image to a display control section 37 according to a calculation result by an insertion length calculation section 46 and an insertion shape image from a scope model generation section 35.

When an insertion section 4b is inserted into an intestinal tract, the intestinal tract is actually significantly deformed. If the intestinal tract is greatly bent, the insertion section 4b is difficult to insert. Therefore, a technique for hanging a distal end of the insertion section 4b in the intestinal tract using a locking force such as suction and causing the insertion section 4b to enter the intestinal tract while towing the insertion section 4b, for example, to deform the intestinal tract in a linear shape may be adopted. The present exemplary embodiment enables display of such a state at the time of actual insertion, to display the body outer shape image in which the intestinal tract model has been deformed according to the insertion length and a shape of the insertion shape image.

For example, the body outer shape image generation section 52 grasps at which position on the intestinal tract model the insertion section 4b is positioned based on the calculated insertion length. The body outer shape image generation section 52 also finds a curvature of each portion of the insertion shape image using information from the scope model generation section 35. The body outer shape image generation section 52 bends an image portion of the intestinal tract model corresponding to the insertion length at an angle corresponding to the curvature of the insertion shape image.

In a sigmoid colon, for example, an intestinal tract model image is bent with a relatively large curvature. When the insertion section 4b is actually inserted, a sigmoid colon portion is deformed in a relatively linear shape. The body outer shape image generation section 52 generates and outputs a body outer shape image in which an image portion of a sigmoid colon has been deformed to have a curvature corresponding to a curvature of an insertion shape image.

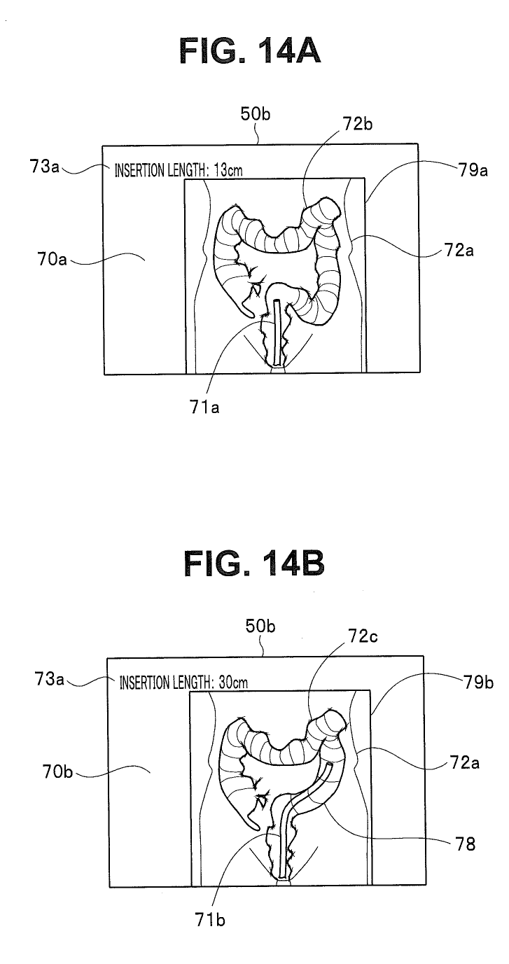

Then, an operation according to the exemplary embodiment thus configured will be described with reference to explanatory diagrams of FIGS. 14A and 14B. FIGS. 14A and 14B are explanatory diagrams each illustrating an inserted state display image displayed on a display screen of a monitor 50.

The body outer shape image generation section 52 acquires information about an insertion length from the insertion length calculation section 46. The body outer shape image generation section 52 also acquires information about an insertion shape image from the scope model generation section 35. The body outer shape image generation section 52 roughly grasps at which position in a body cavity the insertion section 4b is positioned by the insertion length. The body outer shape image generation section 52 deforms a portion of an intestinal tract model image at a site in the body cavity found from the insertion length based on a curvature of the insertion shape image positioned at the site.

For example, it is assumed that the insertion length is relatively short, and a distal end of the insertion section 4b has not reached a sigmoid colon portion. In this case, an inserted state display image 70 illustrated in FIG. 14A, for example, is displayed. The inserted state display image 70 includes an insertion shape image 71a and a detailed body outer shape image 79a. The inserted state display image 70a also includes an insertion length display 73a indicating that the insertion length is 13 cm.

The detailed body outer shape image 79a includes a body outer shape contour image 72a having a shape close to an actual body shape of a human body while including an intestinal tract model image 72b. The insertion shape image 71a indicates into which position in the intestinal tract model image 72b the insertion section 4b has been inserted.

It is assumed that an operator further inserts the insertion section 4b into an intestinal tract. For example, it is assumed that the insertion length becomes 30 cm indicating that the insertion section 4b has passed through the sigmoid colon portion and has reached a descending colon portion. In this case, the body outer shape image generation section 52 deforms the sigmoid colon image portion based on a curvature of each portion of the insertion shape image positioned at the site.

FIG. 14B illustrates an inserted state display image 70b displayed on a display screen 50b in this case. The inserted state display image 70b includes an insertion shape image 71b and a detailed body outer shape image 79b. The inserted state display image 70b also includes an insertion length display 73a indicating that an insertion length is 30 cm.

The body outer shape image 79b includes a body outer shape contour image 72a having a shape close to an actual body shape of a human body while including an intestinal tract model image 72c. The intestinal tract model image 72c includes an image portion 78 having a curvature corresponding to the insertion shape image 71b and deformed into a shape close to a linear shape in a sigmoid colon portion. When the intestinal tract model image 72c including the image portion 78 and the insertion shape image 71b are referred to, it can be easily grasped how an intestinal tract is being deformed while the insertion section 4b is inserted thereto and into which position in an intestinal tract model the insertion section 4b has been inserted.

Accordingly, in the present exemplary embodiment, a similar effect to the effect in the first exemplary embodiment is obtained while the body outer shape image can be deformed and displayed by a result of the insertion length and the insertion shape image, an appropriate body outer shape image corresponding to each scene to be inserted can be displayed, and an inserted state can be easily and reliably confirmed.

Note that the above-described exemplary embodiment has been described as deforming the body outer shape image based on the insertion length and the insertion shape image. However, if a shape after the deformation is known, a plurality of body outer shape images corresponding to the shape after the change may be registered to select the corresponding body outer shape image based on the insertion length and the insertion shape image.

(Modification)

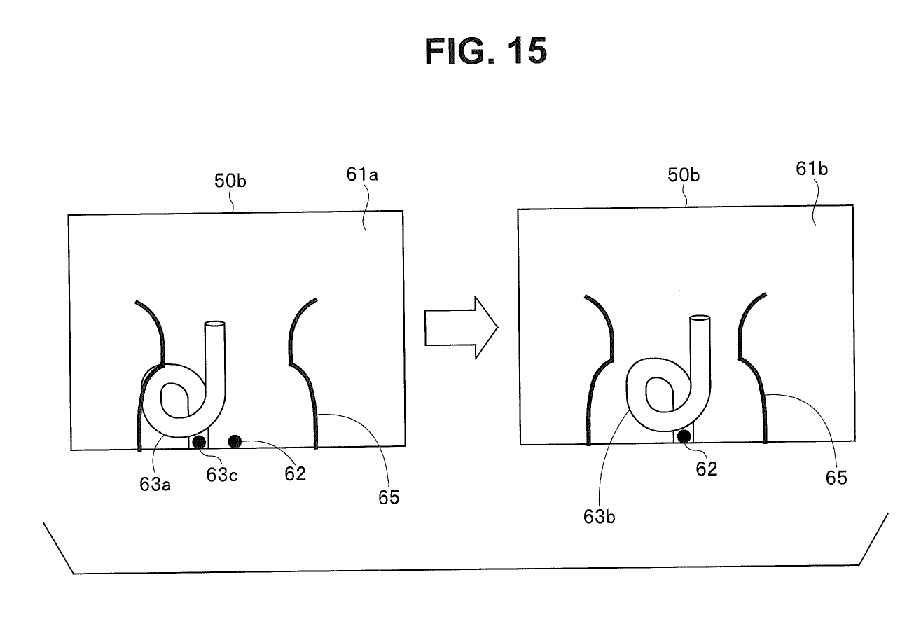

FIG. 15 is an explanatory diagram for describing a modification. Each of the above-described exemplary embodiments has been described as always detecting the anus position of the subject P using the marker 41. However, the anus position may be detected using the marker 41 only at the start of inspection, and the marker 41 may not be used hereafter. When an anus position of a patient changes in this case, a body outer shape image is displayed with the anus position matched with a reference position on a display screen while an insertion shape image is displayed with an anus position different from the actual anus position matched with the reference position. A shift occurs in alignment between the insertion shape image and the body outer shape image.

It is considered that the anus position is positioned within a predetermined plane including an anus position detected at the start of inspection only by the patient changing his/her orientation between a supine position and a lateral position at the time of normal inspection. Therefore, in the modification, assuming that a patient P lies parallel to a longitudinal direction of a bed 6, and an anus position moves within a plane including an anus position detected at the start of inspection and perpendicular to the longitudinal direction of the bed 6 (hereinafter referred to as an insertion position plane), position coordinates of a transmission coil 24 positioned within the plane or in the vicinity of the plane (hereinafter referred to as an anus position coil) is set to anus position coordinates to perform alignment.

A left side of FIG. 15 represents an inserted state display image 61a when the anus position of the patient has changed in the first exemplary embodiment. The inserted state display image 61a includes an insertion shape image 63a and a body outer shape image 65. The body outer shape image 65 is displayed with its anus position matched with a reference position 62 on a display screen 50b. On the other hand, the insertion shape image 63a is displayed with an anus position at the start of inspection different from an actual anus position 63c matched with the reference position 62. A shift occurs in alignment between the insertion shape image 63a and the body outer shape image 65.

On the other hand, in the modification, an inserted state display image 61b illustrated on a right side of FIG. 15 is displayed on a display screen 50b. The inserted state display image 61b includes an insertion shape image 63b and a body outer shape image 65. The body outer shape image 65 is displayed with its anus position matched with a reference position 62 on the display screen 50b.

In the modification, a display control section 37 displays the insertion shape image 63b such that an anus position 63c found by a position of the anus position coil is matched with the reference position 62. That is, the insertion shape image 63b is displayed such that the actual anus position matches the reference position 62, and the insertion shape image 63b and the body outer shape image 65 are displayed in an aligned state.

Accordingly, the modification has an advantage that even when the anus position moves within the insertion position plane, the insertion shape image and the body outer shape image can be displayed in an aligned state.

### Fifth Exemplary Embodiment

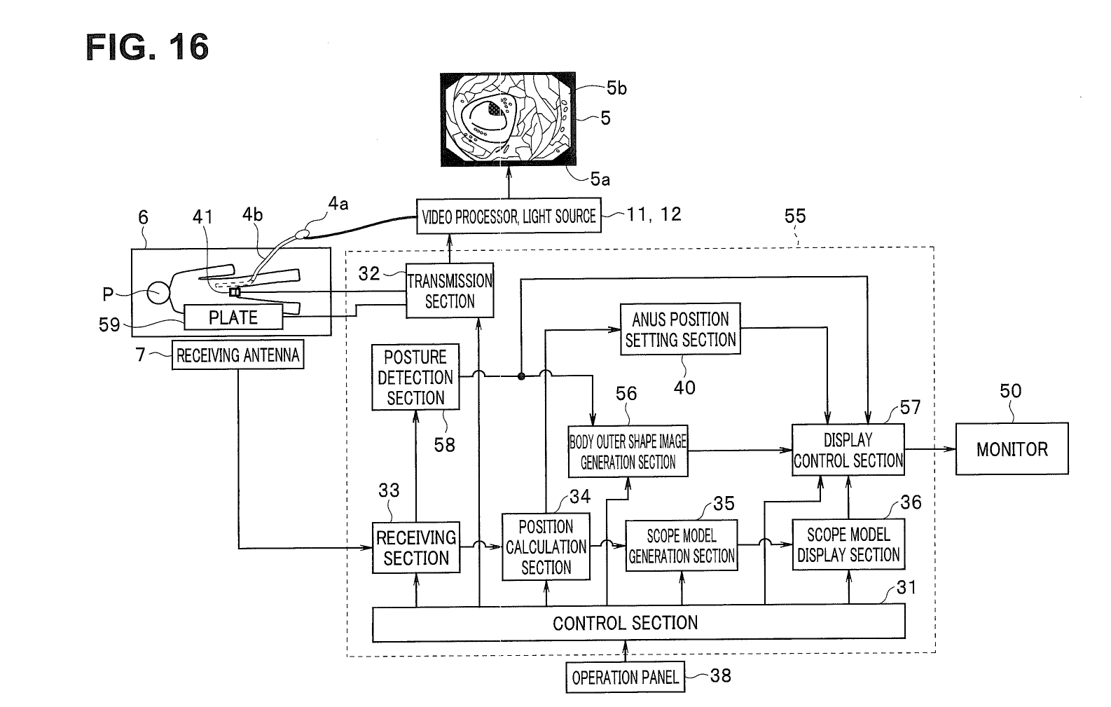

FIG. 16 is a block diagram illustrating a fifth exemplary embodiment. In FIG. 16, similar components to the components illustrated in FIG. 1 are assigned the same reference numerals, and description of the components is omitted. In the above-described modification, if the anus position is within the insertion position plane, the insertion shape image and the body outer shape image can be displayed in an aligned state. However, the anus position of the patient P may actually shift from the insertion position plane found at the start of inspection. Each of the above-described exemplary embodiments premises that the patient P is lying in a state parallel to the longitudinal direction of the bed 6. If the patient P has lain while being inclined with respect to the longitudinal direction of the bed 6, the insertion shape image on the display screen 50b is also displayed while being inclined. Not to change the insertion shape image regardless of a case where the patient P has changed his/her posture between the supine position and the lateral position, an operation for changing a transformation method for coordinate transformation between a measurement coordinate system and a display coordinate system depending on the orientation of the patient P needs to be performed using the operation panel 38 or the like. The present exemplary embodiment solves the problems and makes it possible to automatically display an appropriate inserted state display image.

A control unit 55 in the present exemplary embodiment differs from the control unit 10 illustrated in FIG. 1 in that a body outer shape image generation section 56 and a display control section 57 are adopted instead of the body outer shape image generation section 39 and the display control section 37 while a posture detection section 58 is provided. In the present exemplary embodiment, a plate 59 is adopted.

The plate 59 is a plate-shaped member, for example, and is provided with transmission coils not illustrated in three portions, for example. The transmission coils in the three portions provided in the plate 59 determine a position and an orientation of a plane of the plate 59 (hereinafter referred to as a plate plane). For example, a plane including respective positions of all the transmission coils in the three portions may be set as the plate plane. The plate 59 is affixed and fixed, for example, to a predetermined position of a trunk or the like on a body surface of a patient P. Therefore, if the patient P has changed his/her orientation between a supine position and a lateral position and if the patient P has a predetermined angle to a longitudinal direction of a bed 6, for example, the orientation of the plate plane corresponds to the orientation of the patient P. A positional relationship between a position of the plate plane, e.g., a position of a center of gravity of the plate plane and an anus position of the patient P does not change regardless of a change in the anus position of the patient P. Therefore, even if a marker 41 is used only at the start of inspection, actual anus position coordinates can be found from the position of the plate plane using anus position coordinates found at the start of inspection.

High-frequency sine waves are respectively fed to the transmission coils in the plate 59 from a transmission section 32 in the control unit 10. Each of the transmission coils provided in the plate 59 radiates an electromagnetic wave with a magnetic field to surroundings when the high-frequency sine wave is applied to the transmission coil. A receiving antenna 7 receives the magnetic field generated by the transmission coil in the plate 59 using a sense coil, converts the magnetic field into a voltage signal, and outputs the voltage signal as a detection result. A detection result by the receiving antenna 7 is given to a receiving section 33 in the control unit 10. The receiving section 33 subjects a signal from the receiving antenna 7 to predetermined signal processing such as amplification processing and then outputs the signal to a position calculation section 34. The position calculation section 34 calculates position coordinates, using a position of the receiving antenna 7 as a reference, of each of the transmission coils in the plate 59 from the received signal based on a known position estimation algorithm.

A calculation result of the position coordinates of each of the transmission coils in the plate 59 by the position calculation section 34 is fed to the posture detection section 58. The posture detection section 58 can find the position and the orientation of the plate plane from the position coordinates of each of the transmission coils in the plate 59. The posture detection section 58 outputs the position and the orientation of the plate plane to the body outer shape image generation section 56 and the display control section 57.

The body outer shape image generation section 56 includes display data for a body outer shape image viewed from the side of a front surface of a body (an abdomen) and display data for a body outer shape image viewed from the side of a side surface of the body (a body side) in a memory not illustrated, and is controlled by a control section 31, to selectively output one of a body outer shape image viewed from the side of the abdomen (hereinafter referred to as a front surface body outer shape image), a body outer shape image viewed from the side of a right body side (hereinafter referred to as a right body outer shape image), and a body outer shape image viewed from the side of a left body side (hereinafter referred to as a left body outer shape image) to the display control section 57. Note that the body outer shape image generation section 56 may select one of a plurality of body outer shape images which differ in viewpoints and output the selected body outer shape image to the display control section 57 based on a detection result by the posture detection section 58.

The display control section 57 is given display data for an insertion shape image from a scope model display section 36 and is given the display data for the body outer shape image from the body outer shape image generation section 56 upon being controlled by the control section 31, to display an inserted state display image on a display screen 50b of a monitor 50. In this case, the display control section 57 changes transformation from a measurement coordinate system to a display coordinate system based on the detection result by the posture detection section 58, to change a position and an orientation of the insertion shape image.

That is, the display control section 57 can display the insertion shape image always viewed from the same direction even when the patient P is in any posture between the supine position and the lateral position by rotating the insertion shape image depending on the orientation of the plate plane.

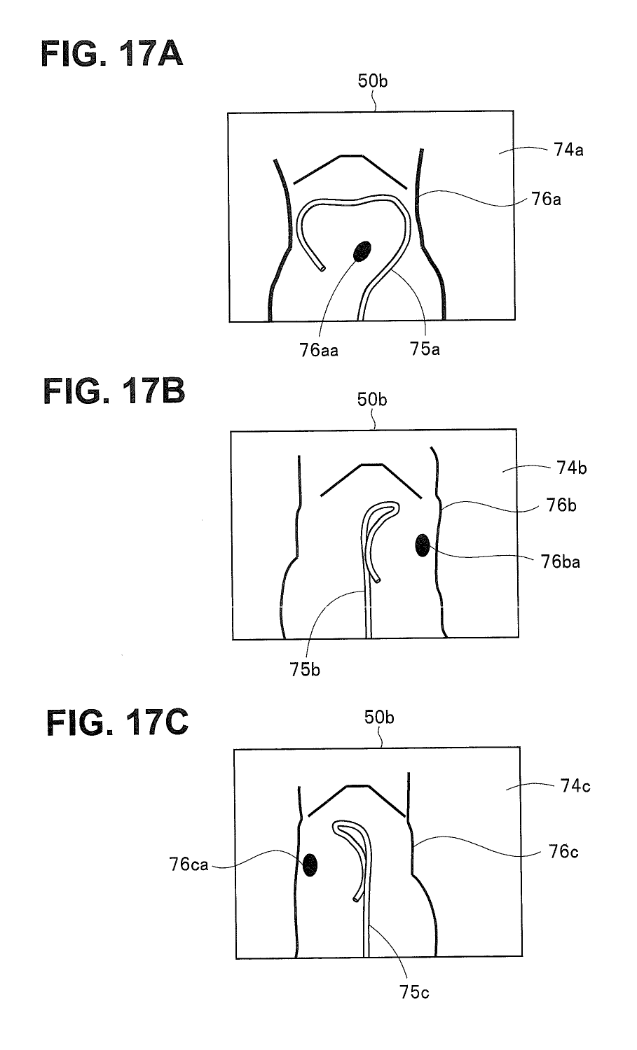

Then, an operation according to the exemplary embodiment thus configured will be described with reference to explanatory diagrams of FIGS. 17A to 17C. FIGS. 17A to 17C illustrate an inserted state display image displayed on the display screen of the monitor 50.

The posture detection section 58 detects a position and an orientation of the plate plane of the plate 59 and outputs a detection result to the body outer shape image generation section 56 and the display control section 57. The body outer shape image generation section 56 is controlled by the control section 31, to selectively output one of a front surface body outer shape image, a right body outer shape image, and a left body outer shape image to the display control section 57. The display control section 57 displays an inserted state display image obtained by aligning and synthesizing an anus position, for example, of an insertion shape image and an anus position of a body outer shape image with a reference position of the display screen 50b on the display screen 50b of the monitor 50.

It is assumed that an inserted state display image 74a illustrated in FIG. 17A is displayed on the display screen 50b of the monitor 50 when the patient P is in a state of a supine position. That is, the display control section 57 synthesizes a front surface body outer shape image 76a from the body outer shape image generation section 56 and an insertion shape image 75a viewed from the side of an abdomen of the patient P. The body outer shape image 76a also includes an image portion of a navel portion 76aa.

It is assumed that the patient P changes his/her posture to a state of a lateral position in this state. In this case, a trajectory of each of coil positions detected by the position calculation section 34 is obtained by rotating a trajectory of the coil position detected in the state of the supine position using a straight line passing through the anus position and in the longitudinal direction of the bed 6 as an axis. To reverse the rotation, the display control section 57 performs coordinate transformation for the insertion shape image from the scope model display section 36 depending on an inclination of the plate plane from the posture detection section 58. As a result, the display control section 57 continues to display the inserted state display image 74a illustrated in FIG. 17A even when the patient P has changed the posture to the state of the lateral position.

It is assumed that a right body outer shape image 76b illustrated in FIG. 17B is outputted from the body outer shape image generation section 56. In this case, the display control section 57 displays an inserted state display image 74b illustrated in FIG. 17B. The inserted state display image 74b includes a right body outer shape image 76b and an insertion shape image 75b. The body outer shape image 76b also includes an image portion of a navel portion 76ba.

In this case, the display control section 57 also performs coordinate transformation to rotate the insertion shape image from the scope model display section 36 depending on an inclination of the plate plane from the posture detection section 58. As a result, the inserted state display image 74b illustrated in FIG. 17B continues to be displayed regardless of the posture of the patient P.

It is assumed that a left body outer shape image 76c illustrated in FIG. 17C is outputted from the body outer shape image generation section 56. In this case, the display control section 57 displays an inserted state display image 74c illustrated in FIG. 17C. The inserted state display image 74c includes a right body outer shape image 76c and an insertion shape image 75c. The body outer shape image 76c also includes an image portion of a navel portion 76ca.

In this case, the display control section 57 also performs coordinate transformation to rotate the insertion shape image from the scope model display section 36 depending on an inclination of the plate plane from the posture detection section 58. As a result, the inserted state display image 74c illustrated in FIG. 17C continues to be displayed regardless of the posture of the patient P.

Note that, although an example in which the coordinate transformation of the insertion shape image is controlled by detecting the position and the orientation of the plate plane to align and display the body outer shape image and the insertion shape image which are the same in viewpoints has been described above, the body outer shape image and the insertion shape image which respectively change in viewpoints depending on the posture of the patient P can also be aligned and displayed by detecting the position and the orientation of the plate plane and changing a type of the body outer shape image depending on a detection result.

Note that the display control section 57 can display, even when the patient P has lain while being inclined at the predetermined angle to the longitudinal direction of the bed 6, a similar insertion shape image to when the patient P has always laid parallel to the longitudinal direction of the bed 6, by rotating the insertion shape image around the reference position of the display screen 50b depending on the inclination of the plate plane with respect to the longitudinal direction of the bed 6.

Similarly, the display control section 57 can display, even when the anus position of the patient P has shifted from the insertion position plane found at the start of inspection, the inserted state display image obtained by always correctly aligning the insertion shape image and the body outer shape image by matching the anus position coordinates which have changed depending on the change in the position of the plate plane with the reference position of the display screen 50b.

Accordingly, in the present exemplary embodiment, a similar effect to the effect in the first exemplary embodiment is obtained while the inserted state display image obtained by aligning the insertion shape image and the body outer shape image can be displayed regardless of the position and the orientation of the patient by detecting the position and the orientation of the plate plane using the plate to be fixed to the patient to change the transformation method for the coordinate transformation.

### Sixth Exemplary Embodiment

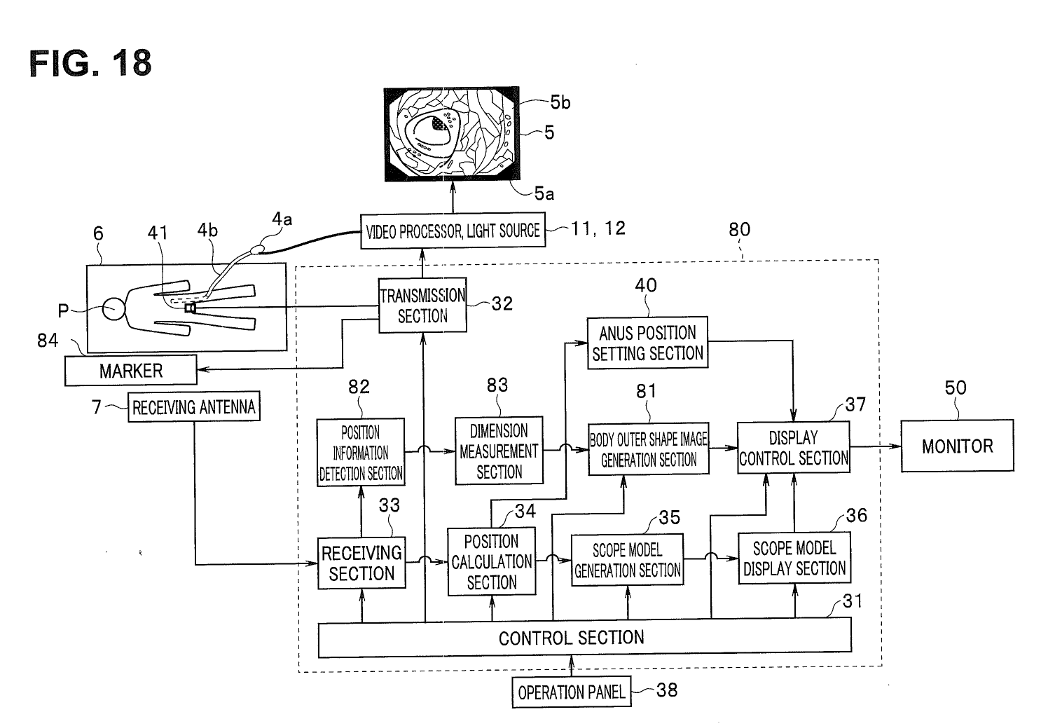

FIG. 18 is a block diagram illustrating a sixth exemplary embodiment. In FIG. 18, similar components to the components illustrated in FIG. 1 are assigned the same reference numerals, and description of the components is omitted. The present exemplary embodiment makes it possible to change a body outer shape image to a size corresponding to a dimension of a patient.

A control unit 80 in the present exemplary embodiment differs from the control unit 10 illustrated in FIG. 1 in that a body outer shape image generation section 81 is adopted instead of the body outer shape image generation section 39 while being provided with a position information detection section 82 and a dimension measurement section 83. In the present exemplary embodiment, a marker 84 is adopted.

The marker 84 contains a transmission coil not illustrated, like the marker 41, and a high-frequency sine wave is applied to the transmission coil from a transmission section 32. The marker 84 generates a magnetic field when the high-frequency sine wave is applied thereto from the transmission section 32. The magnetic field is received by a receiving antenna 7, and a detection result by the receiving antenna 7 is fed to the position information detection section 82 via a receiving section 33. The position information detection section 82 acquires position coordinates of the marker 84 in a measurement coordinate system by a similar operation to an operation of a position calculation section 34, and outputs the acquired position coordinates to the dimension measurement section 83.

In the present exemplary embodiment, the marker 84 is used to find a dimension of a patient. For example, markers 84 are respectively arranged in a plurality of portions of a trunk of the patient to find the dimension of the patient. For example, position coordinates are acquired with the markers 84 arranged in a total of three portions, i.e., respective positions of two portions of both body sides at a height of a navel portion and an anus position.

The dimension measurement section 83 is given position coordinates in each of the plurality of portions of the trunk of the patient, and measures the dimension of the patient using the position coordinates. For example, the dimension measurement section 83 may find a transverse width and a longitudinal length, for example, of the trunk of the patient. A measurement result by the dimension measurement section 83 is given to the body outer shape image generation section 81.

The body outer shape image generation section 81 holds display data for a body outer shape image in a memory not illustrated, and is controlled by a control section 31, to enlarge or reduce the body outer shape image in longitudinal and transverse directions to change scaling based on the measurement result by the dimension measurement section 83, and then output the body outer shape image to the display control section 37.

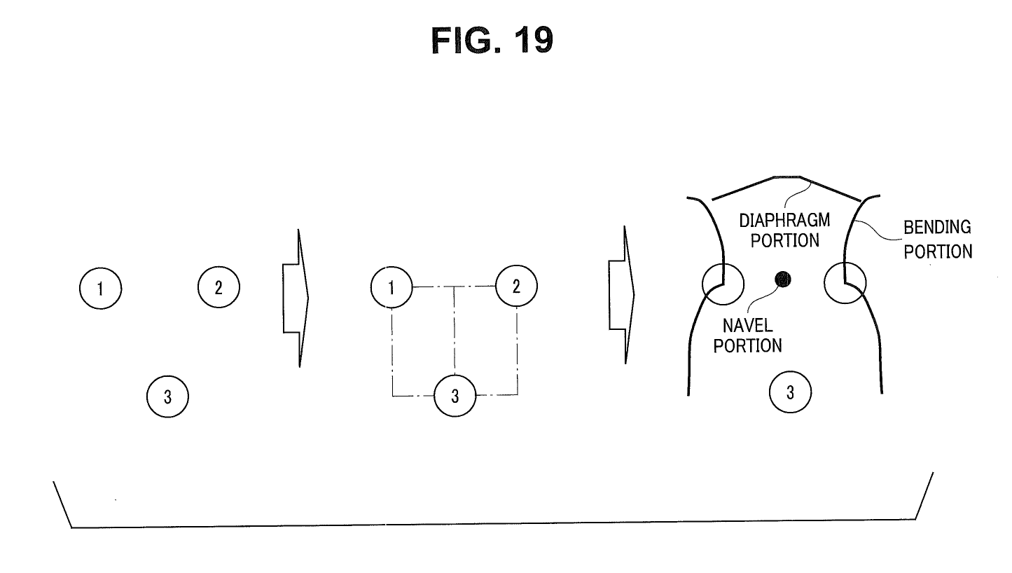

FIG. 19 is an explanatory diagram illustrating an example of a method for finding a dimension of a patient and a method for generating a body outer shape image. A left side, a center, and a right side of FIG. 19 respectively illustrate a detection result of a position of a body surface of a patient P by the marker 84, measurement of the dimension by the dimension measurement section 83, and scaling of the body outer shape image.

For example, a circled number 1, a circled number 2, and a circled number 3 on the left side of FIG. 19 respectively indicate a position on a right body side at a height of a navel portion of the patient P, a position on a left body side at the height of the navel portion of the patient P, and an anus position of the patient P. Respective information about the positions is fed to the dimension measurement section 83.

The dimension measurement section 83 finds a length (a trunk width) between the position on the right body side and the position on the left body side respectively indicated by the circled numbers 1 and 2, as illustrated at the center of FIG. 19, based on input positional information. The dimension measurement section 83 also finds a length (a trunk length) from a straight line connecting the position on the right body side and the position on the left body side respectively indicated by the circled numbers 1 and 2 to the anus position indicated by the circled number 3. Respective information about the trunk width and the trunk length are fed to the body outer shape image generation section 81.

The body outer shape image stored in the body outer shape image generation section 81 includes respective image portions of a bending portion representing a contour of the body, a diaphragm portion representing a diaphragm, and a navel portion representing a position of a navel as illustrated at the right side of FIG. 19, for example. The body outer shape image generation section 81 enlarges or reduces a transverse width of the body outer shape image such that a spacing between portions (circled portions), at a height position of the navel portion, of the bending portion representing both the body sides matches the trunk width. The body outer shape image generation section 81 also enlarges or reduces the longitudinal direction of the body outer shape image such that a spacing from the navel portion to the anus position indicated by the circled number 3 matches the trunk length. The right side of FIG. 19 represents the body outer shape image after the enlargement or reduction.

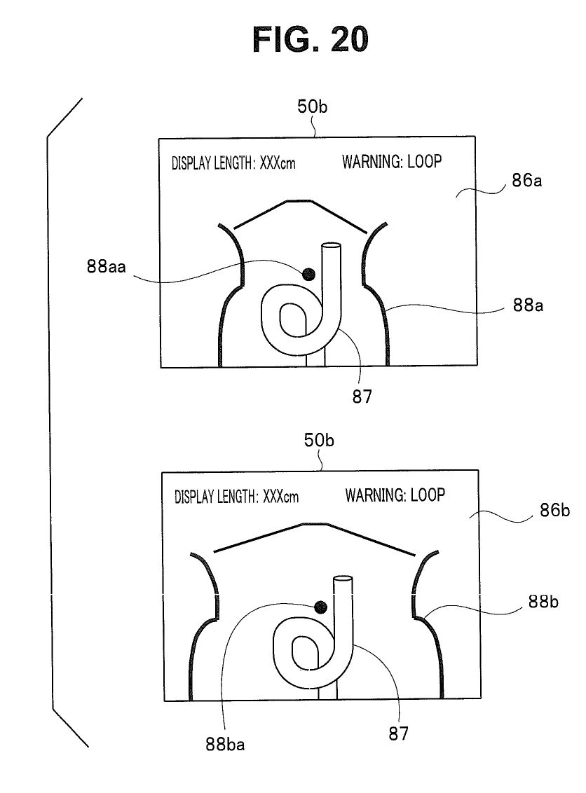

Then, an operation according to the exemplary embodiment thus configured will be described with reference to an explanatory diagram of FIG. 20. FIG. 20 illustrates an inserted state display image displayed on a display screen of a monitor 50.

An operator arranges the marker 84 in each of the portions of the patient P to find the dimension of the patient P. The position information detection section 82 acquires positional information of the markers 84 respectively arranged in the portions of the patient P and outputs the acquired positional information to the dimension measurement section 83. The dimension measurement section 83 finds the dimensions of the patient P respectively based on the inputted plurality of pieces of positional information. For example, the dimension measurement section 83 finds the trunk width and the trunk length of the patient P and outputs the found trunk width and trunk length to the body outer shape image generation section 81.

The body outer shape image generation section 81 enlarges or reduces the stored body outer shape image to correspond to the inputted dimension of the patient P, to generate a body outer shape image which matches the dimension of the patient P and output the generated body outer shape image to the display control section 37.

For example, it is assumed that an insertion section 4b is inserted into a body cavity of a relatively thin patient. In this case, the body outer shape image generation section 81 generates a body outer shape image in which a trunk width is relatively narrow depending on a measurement result of the dimension of the patient. An upper side of FIG. 20 illustrates an inserted state display image 86a displayed on the display screen 50b of the monitor 50 in this case. The inserted state display image 86a includes an insertion shape image 87 and a body outer shape image 88a. The body outer shape image 88a also includes an image portion of a navel portion 88aa.

A size of the body outer shape image 88a corresponds to a size obtained by finding positional information of each of the portions of the patient P, and corresponds to a size of the insertion shape image 87 generated based on positional information of each of transmission coils 24 in the insertion section 4b. Therefore, the insertion shape image 87 and the body outer shape image 88a in the inserted state display image 86a make it possible to accurately grasp into which position in the body cavity of the patient P the insertion section 4b has been inserted.

For example, it is assumed that the insertion section 4b is inserted into a body cavity of a relatively fat patient. In this case, the body outer shape image generation section 81 generates a body outer shape image in which a trunk width is relatively wide depending on a measurement result of the dimension of the patient. A lower side of FIG. 20 illustrates an inserted state display image 86b displayed on the display screen 50b of the monitor 50 in this case. The inserted state display image 86b includes an insertion shape image 87 and a body outer shape image 88b. The body outer shape image 88b also includes an image portion of a navel portion 88ba.

A size of the body outer shape image 88b corresponds to a size obtained by finding the positional information of each of the portions of the patient P, and corresponds to the size of the insertion shape image 87. Therefore, the insertion shape image 87 and the body outer shape image 88b in the inserted state display image 86b make it possible to accurately grasp into which position in the body cavity of the patient P the insertion section 4b has been inserted.

Accordingly, in the present exemplary embodiment, a similar effect to the effect in the first exemplary embodiment is obtained while the body outer shape image of the size corresponding to the size of the patient can be displayed. As a result, the inserted state display image including the insertion shape image and the body outer shape image makes it possible to accurately grasp into which position in the body cavity of the patient the insertion section has been inserted.

Note that, although an example in which the body outer shape image is enlarged or reduced based on the trunk width and the trunk length has been described, the present exemplary embodiment is not limited to the trunk width and the trunk dimension. The body outer shape image may be enlarged or reduced using the dimension of each of the portions of the patient, e.g., a thickness of an abdomen, for example.

### Seventh Exemplary Embodiment

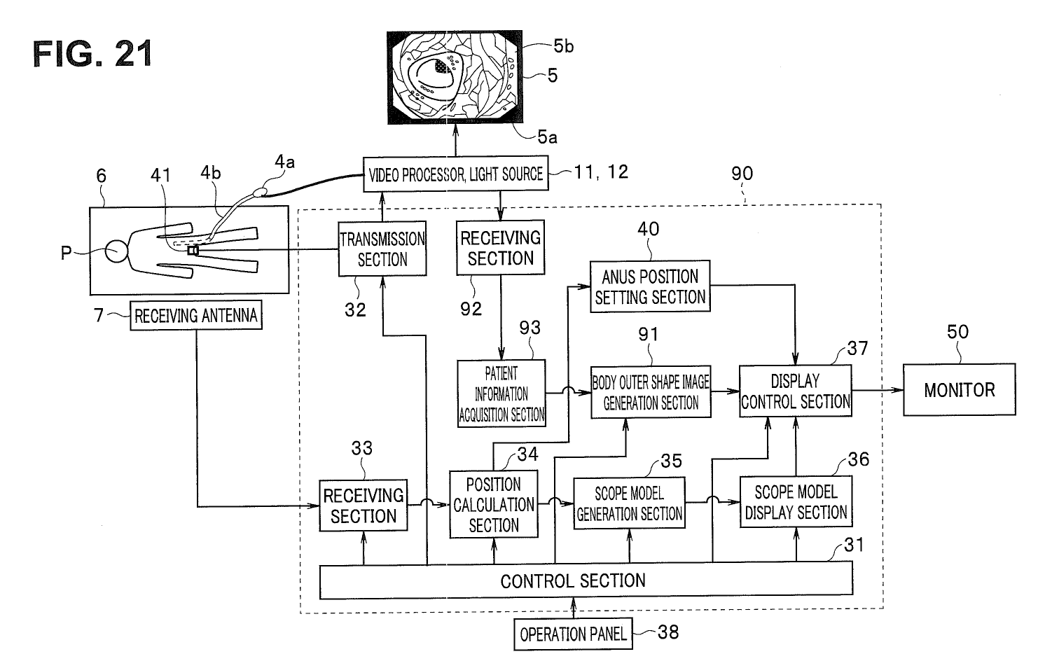

FIG. 21 is a block diagram illustrating a seventh exemplary embodiment. In FIG. 21, similar components to the components illustrated in FIG. 1 are assigned the same reference numerals, and description of the components is omitted. In the present exemplary embodiment, a body outer shape image having a shape corresponding to a body shape of a patient is displayed.

A control unit 90 in the present exemplary embodiment differs from the control unit 10 illustrated in FIG. 1 in that a body outer shape image generation section 91 is adopted instead of the body outer shape image generation section 39 while being provided with a receiving section 92 and a patient information acquisition section 93.

Patient information about a patient P who undergoes an inspection, for example, is registered in a video processor 12. Examples of the patient information include a patient ID, a patient name, a sex, a height, a weight, and a BMI (body mass index) value.

The receiving section 92 is controlled by a control section 31, to receive the patient information from the video processor 12 at a predetermined timing and feed the received patient information to the patient information acquisition section 93. The patient information acquisition section 93 acquires the patient information and outputs information about the body shape of the patient (hereinafter referred to as body shape information), e.g., body shape information such as a height, a weight, and a BMI value to the body outer shape image generation section 91.

The body outer shape image generation section 91 holds display data for body outer shape images respectively corresponding to a plurality of types of body shapes in a memory not illustrated, and is controlled by the control section 31, to select, based on the body shape information from the patient information acquisition section 93, the body outer shape image corresponding to the corresponding body shape and output the selected body outer shape image to a display control section 37. For example, the body outer shape image generation section 91 holds various types of body outer shape images respectively corresponding to sections of the body shape, the height, and the like, e.g., an A body shape, a Y body shape, an AB body shape, a B body shape, an S size, an M size, and an L size.

For example, the body outer shape image generation section 91 may judge to which of the S to L sizes the body outer shape image corresponds based on information about the height among the body shape information and judge to which of the A body shape to the B body shape the body outer shape image corresponds based on information about the weight or the BMI among the body shape information.

For example, the body outer shape image generation section 91 may store a plurality of types of body outer shape images which differ in trunk width and trunk length, and determine the trunk width based on the information about the weight or the BMI and determine the trunk length based on the information about the height, to select and output the corresponding body outer shape image.

Then, an operation according to the exemplary embodiment thus configured will be described with reference to an explanatory diagram of FIG. 20. FIG. 20 illustrates an inserted state display image displayed on a display screen of a monitor 50.

The control section 31 controls the receiving section 92, to receive patient information of the patient P from the video processor 12. The patient information acquisition section 93 acquires the patient information, and outputs the body shape information to the body outer shape image generation section 91.

The body outer shape image generation section 91 selects one of stored body outer shape images to correspond to the inputted body shape information of the patient P, and outputs the body outer shape image which matches the body shape or the like of the patient P to the display control section 37.

For example, it is assumed that an insertion section 4b is inserted into a body cavity of a relatively thin patient. In this case, the body outer shape image generation section 91 generates a body outer shape image in which a trunk width is relatively narrow depending on the body shape information of the patient P. In this case, an inserted state display image 86a illustrated on an upper side of FIG. 20 is displayed on the display screen 50b of the monitor 50, for example.

For example, it is assumed that the insertion section 4b is inserted into a body cavity of a relatively fat patient. In this case, the body outer shape image generation section 91 generates a body outer shape image in which a trunk width is relatively wide depending on the body shape information of the patient P. In this case, an inserted state display image 86b illustrated in a lower side of FIG. 20 is displayed on the display screen 50b of the monitor 50, for example.

Respective shapes of body outer shape images 88a and 88b are based on the body shape information of the patient P, and correspond to a size of an insertion shape image 87. Therefore, even if the patient P having any body shape is inspected, the inserted state display images 86a and 86b make it possible to accurately grasp into which position in the body cavity of the patient P the insertion section 4b has been inserted.

Accordingly, in the present exemplary embodiment, a similar effect to the effect in the first exemplary embodiment is obtained while the body outer shape image having the body shape corresponding to the body shape information of the patient can be displayed. As a result, the inserted state display image including the insertion shape image and the body outer shape image makes it possible to accurately grasp into which position in the body cavity of the patient the insertion section has been inserted.

(Modification)

Although the body outer shape image of the size corresponding to the dimension of the patient P has been displayed by measuring the dimension of the patient P and enlarging or reducing the body outer shape image based on the measurement result of the dimension in the above-described sixth embodiment, a body outer shape image having a body shape corresponding to body shape information of a patient P may be displayed by acquiring patient information and enlarging or reducing the body outer shape image based on the body shape information of the patient in combination with the seventh exemplary embodiment.

Although the body outer shape image corresponding to the body shape corresponding to the body shape information of the patient P has been displayed by acquiring the patient information of the patient P and selecting the body outer shape image corresponding to the body shape based on the body shape information of the patient in the above-described seventh exemplary embodiment, a body outer shape image of a size corresponding to a dimension of a patient P may be displayed by measuring the dimension of the patient P and selecting a body outer shape image of a corresponding size based on a measurement result of the dimension in combination with the sixth exemplary embodiment.

An insertion section may have a loop shape in a body cavity, as described above. In this case, an operator recognizes that the insertion section has a loop shape from an insertion shape image on a display screen of a monitor. Note that the insertion shape image in a loop portion becomes a display in which an image portion on a front side of a viewpoint and an image portion on a back side of the viewpoint overlap each other.

However, the insertion shape image displayed on the monitor has a problem that a depth is not easily felt and it is not easily recognized in what way the insertion section is looped because a change in color is small between the image portion on the front side of the viewpoint and the image portion on the back side of the viewpoint in the loop portion.

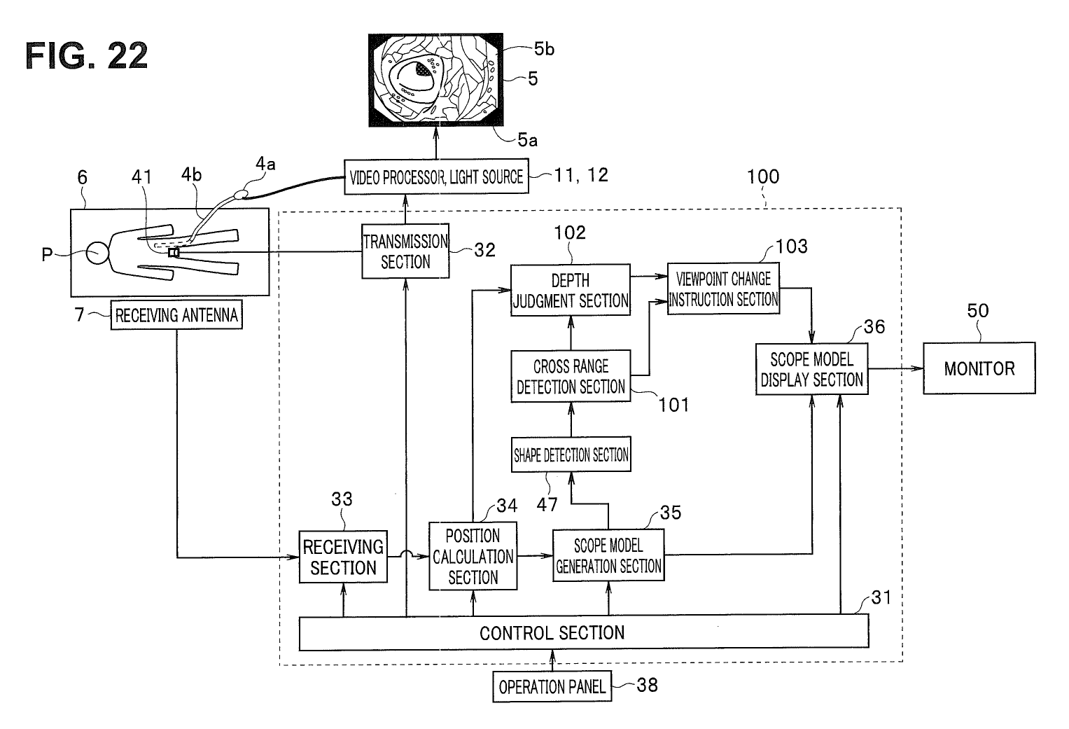

FIG. 22 is a block diagram illustrating an example in which such a problem can be solved. In FIG. 22, similar components to the components illustrated in FIG. 7 are assigned the same reference numerals, and description of the components is omitted.

A control unit 100 includes a cross range detection section 101, a depth judgment section 102, and a viewpoint change instruction section 103. The cross range detection section 101 is given a detection result of a loop shape from a shape detection section 47, to detect a cross range of a loop shape portion and output a detection result to the viewpoint change instruction section 103. The depth judgment section 102 is given positional information for each of portions in an insertion section 4b from a position calculation section 34, to judge a depth of the each portion and output a judgment result to the viewpoint change instruction section 103.

The viewpoint change instruction section 103 generates a control signal for changing a viewpoint and gives the generated control signal to a scope model display section 36 based on the detection result of the cross range by the cross range detection section 101 and the depth judgment result by the depth judgment section 102. For example, the viewpoint change instruction section 103 may enlarge and display a crossover point in a loop portion and generate a control signal for displaying the crossover point at a center of a screen.

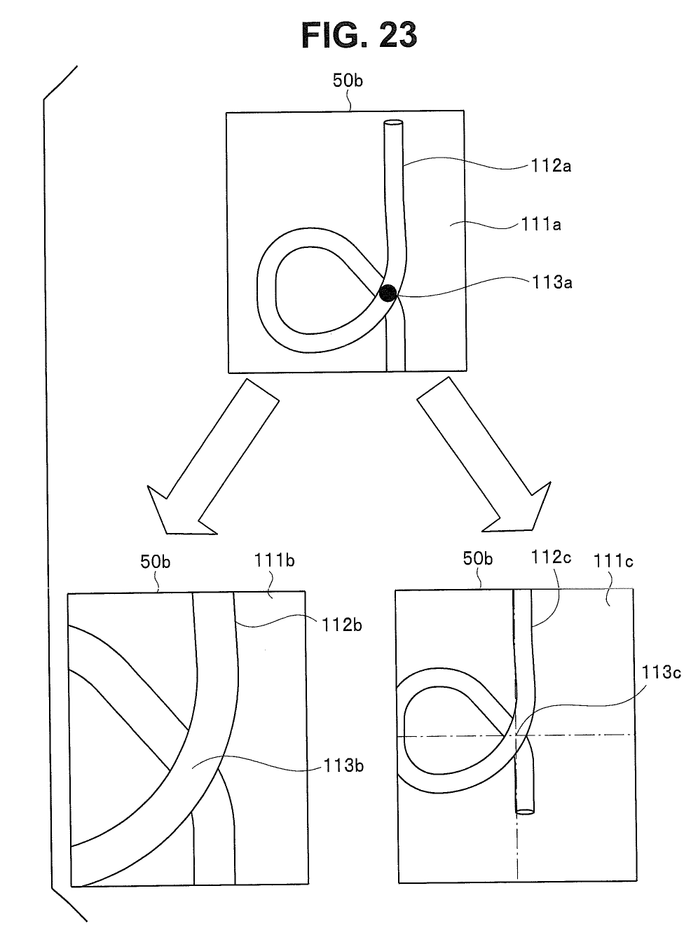

FIG. 23 is an explanatory diagram illustrating an example of an insertion shape image displayed on a display screen of a monitor 50 in this case.

An insertion shape image 111a in FIG. 23 represents an insertion shape which has not been subjected to viewpoint change, and an image 112a representing an insertion shape is displayed. For example, in the image 112a, an anus position of a patient P is matched with a lower end of a display screen 50b. Note that a black circle in FIG. 23 represents a crossover point 113a between an insertion section on a front side of a viewpoint and an insertion section on a back side of the viewpoint.

The viewpoint change instruction section 103 displays a predetermined range including the crossover point in an enlarged manner on the display screen 50b, for example. An insertion shape image 111b in FIG. 23 represents an insertion shape which has been subjected to such enlargement display, and an image 112b representing an insertion shape is displayed. In the enlarged insertion shape image 111b, a portion of a crossover point 113b can be easily viewed by being enlarged, and an image on a front side of a viewpoint and an image on a back side of the viewpoint can also be easily recognized on the monitor.

For example, the viewpoint change instruction section 103 displays the crossover point in the loop portion at the center of the display screen 50b. An insertion shape image 111c in FIG. 23 represents an insertion shape which has been subjected to such viewpoint change, and an image 112c representing an insertion shape is displayed. In the insertion shape image 111c, a portion of a crossover point 113c can be easily viewed by being arranged at a center of the screen, and an image on a front side of a viewpoint and an image on a back side of the viewpoint can also be easily recognized on the monitor.

An apparatus illustrated in FIG. 22 makes it possible to improve visibility of the portion of the crossover point in the insertion shape image displayed on the monitor and makes it easy to recognize the image portion on the front side of the viewpoint and the image portion on the back side of the viewpoint in the loop portion. As a result, an effect of easily recognizing in what way the insertion section has been looped is obtained.

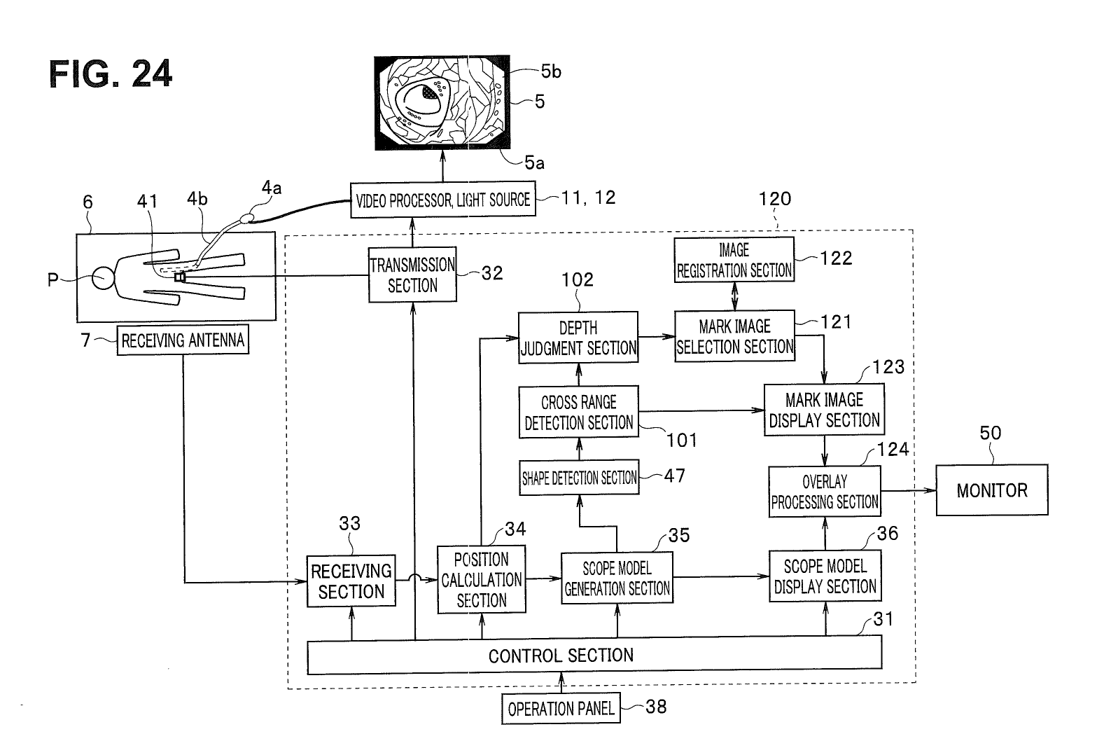

FIG. 24 is a block diagram illustrating another example in which a problem that a loop portion is difficult to confirm can be solved. In FIG. 24, similar components to the components illustrated in FIG. 22 are assigned the same reference numerals, and description of the components is omitted.

A control unit 120 differs from the control unit 100 illustrated in FIG. 22 in that a mark image selection section 121, an image registration section 122, a mark image display section 123, and an overlay processing section 124 are adopted instead of the viewpoint change instruction section 103.

In an example illustrated in FIG. 24, a mark image for making it easy to confirm a loop portion is displayed, and the mark image is registered in the image registration section 122. The mark image selection section 121 selects one or a plurality of mark images among mark images registered in the image registration section 122 and outputs the selected mark image or images to the mark image display section 123 according to a judgment result by a depth judgment section 102.

The mark image display section 123 generates display data for a mark image display region in which the mark image is displayed on a display screen 50b of a monitor 50. The mark image display section 123 is given a detection result by a cross range detection section 101, and sets the mark image display region in the vicinity of a crossover point in the loop portion.

The overlay processing section 124 displays an insertion shape image from a scope model display section 36 and an image of the mark image display region in a superimposed manner on the display screen of the monitor 50 based on the inputted display data.

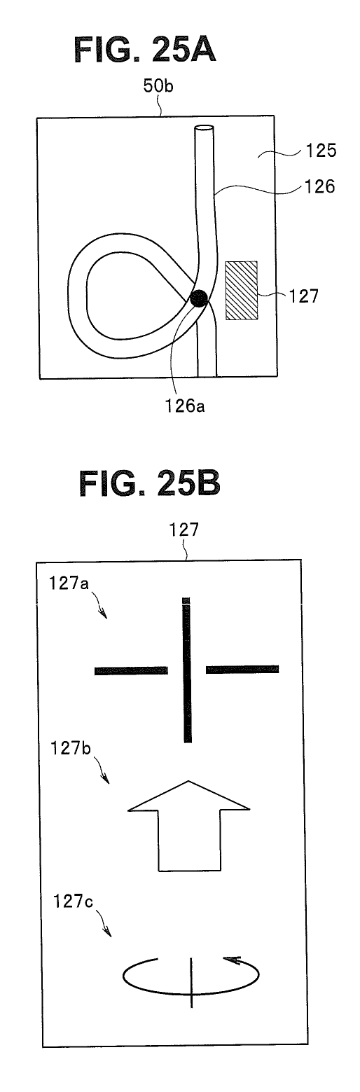

FIGS. 25A and 25B are explanatory diagrams each illustrating an example of an insertion shape image displayed on the display screen of the monitor 50 in this case.

FIG. 25A illustrates an inserted state display image 125 displayed on the display screen 50b. The inserted state display image 125 includes an insertion shape image 126. Further, the inserted state display image 125 is provided with a mark image display region 127. Note that illustration of a mark image in the mark image display region 127 is omitted in FIG. 25A to simplify the drawing.

FIG. 25B illustrates an example of mark images displayed in the mark image display region 127. Although three types of mark images are displayed in the example illustrated in FIG. 25B, one or more mark images may be displayed.

A mark image 127a in FIG. 25B includes a divided linear image extending in a transverse direction and a linear image extending in a longitudinal direction arranged in a portion obtained by the division. The mark image 127a indicates that an image portion extending in the longitudinal direction and an image portion extending in the transverse direction in the insertion shape image 126 are respectively an image on a front side of a viewpoint and an image on a back side of the viewpoint by the linear image in the transverse direction being divided.

A mark image 127b in FIG. 25B indicates in which direction the image portion on the front side of the viewpoint extends by an arrow direction. In an example illustrated in FIG. 25B, the mark image 127b indicates that the image extending in the longitudinal direction in the insertion shape image 126 is the image on the front side of the viewpoint.

A mark image 127c in FIG. 25B indicates a twisting direction of the insertion section for solving a loop. In the example illustrated in FIG. 25B, the mark image 127c indicates that a loop in an insertion section 4b represented by the insertion shape image 126 is eliminated by rotating the insertion section rightward.

Accordingly, the apparatus illustrated in FIG. 24 can make it easy to recognize which image portion in the portion of the crossover point in the insertion shape image is on the front side of the viewpoint or the back side of the viewpoint by using the mark images superior in visibility. In the apparatus illustrated in FIG. 24, the twisting direction for solving the loop can also be easily grasped.

Note that, although the insertion shape image illustrated in each of the drawings is represented only by a contour line, an actual insertion shape image displayed on the monitor is a grayscale image which does not have a contour line but corresponds to a shape of the insertion section. Accordingly, it is considered that the portion of the crossover point in the loop portion is difficult to recognize. Therefore, an insertion shape image a contour line of which is emphasized may be displayed on the display screen of the monitor. The contour line makes it easy to recognize the portion of the crossover point in the loop portion.

Note that each of the sections constituting each of the control units 10, 45, 48, 51, 55, 80, 90, 100, and 120 in the above-described exemplary embodiments may be configured as an individual electronic circuit, or may be configured as a circuit block in an integrated circuit. The control units may be each configured to include one or more CPUs. Alternatively, the control units may each read a program for executing a function of each of the sections from a storage medium such as a memory while performing an operation corresponding to the read program.

The present exemplary embodiment are not limited to the above-described exemplary embodiments as they are but can be embodied by deforming components in an implementation stage without departing from the intended scope. Various embodiments can be formed by appropriate combinations of the plurality of components disclosed in the above-described embodiments. For example, some of all the components described in the embodiments may be deleted. Further, the components over the different exemplary embodiments may be combined, as needed.

What is claimed is:

1. An endoscope insertion shape observation apparatus comprising:
a probe configured to be inserted into a subject; and
a processor programmed to:
detect an insertion shape of the insertion section based on position coordinates of the probe;
generate an insertion shape image representing the detected insertion shape of the probe;
generate a body outer shape image representing a body outer shape of the subject;
simultaneously display, on a display screen, the generated insertion shape image and the generated body outer shape image in a positional relationship corresponding to a positional relationship of the probe in a body cavity of the subject; and
switch between display or non-display of the body outer shape image or a type of the body outer shape image depending on an insertion position in the body cavity of the subject of the probe.

2. An endoscope insertion shape observation apparatus comprising:
a probe configured to be inserted into a subject; and
a processor configured to:
detect an insertion shape of the probe based on position coordinates of the probe;
generate an insertion shape image representing the detected insertion of the probe;
generate a body outer shape image representing a body outer shape of the subject;
simultaneously display, on a display screen, the generated insertion shape image and the generated body outer shape image in a positional relationship corresponding to a positional relationship of the probe in a body cavity of the subject; and
switch between display or non-display of the body outer shape image or a type of the body outer shape image depending on whether the probe has a predetermined shape in the body cavity of the subject.

3. The endoscope insertion shape observation apparatus according to claim 1, wherein the processor is programmed to:
match a position of the insertion shape image corresponding to a reference position of the subject and a position of the body outer shape image corresponding to the reference position of the subject with a display reference position on the display screen.

4. The endoscope insertion shape observation apparatus according to claim 1, wherein the processor is programmed to:
change a viewpoint of the body outer shape image in response to an operation by an operator.

5. The endoscope insertion shape observation apparatus according to claim 1, wherein the processor is programmed to:
change a display position on the display screen of the body outer shape image in response to an operation by an operator.

6. The endoscope insertion shape observation apparatus according to claim 1, wherein the processor is programmed to:
change an inclination of a display on the display screen of the body outer shape image in response to an operation by an operator.

7. The endoscope insertion shape observation apparatus according to claim 1, wherein the processor is programmed to:
change a size of a display on the display screen of the body outer shape image in response to an operation by an operator.

8. The endoscope insertion shape observation apparatus according to claim 1, wherein
the generated body outer shape image includes a schematic body outer shape image representing an outer shape contour of the subject and a detailed body outer shape image including internal organs in the body cavity of the subject.

9. The endoscope insertion shape observation apparatus according to claim 1, wherein the processor is programmed to:
deform the body outer shape image according to an insertion length and an insertion shape image, in the body cavity of the subject, of the probe.

10. The endoscope insertion shape observation apparatus according to claim 1, wherein the processor is programmed to:
judge a position and an orientation of the subject, and
match a position of the insertion shape image corresponding to a reference position of the subject and a position of the body outer shape image corresponding to the reference position of the subject with a display reference position on the display screen based on a detection result of the position and the orientation.

11. The endoscope insertion shape observation apparatus according to claim 10, wherein the processor is programmed to:
rotate either one of the insertion shape image and the body outer shape image based on the detection result of the position and the orientation.

12. The endoscope insertion shape observation apparatus according to claim 1, wherein the processor is programmed to:
generate the body outer shape image while changing a size of the body outer shape image depending on a dimension of the subject.

13. The endoscope insertion shape observation apparatus according to claim 1, further comprising a memory storing a plurality of body outer shape images depending on a body shape of the subject, wherein
the processor is programmed to select the body outer shape image from the stored plurality of body outer shape images corresponding to the body shape of the subject.

14. The endoscope insertion shape observation apparatus according to claim 1, wherein the processor is programmed to:
change a size of the body outer shape image depending on a body shape of the subject.

15. The endoscope insertion shape observation apparatus according to claim 1, further comprising a memory storing a plurality of body outer shape images depending on a dimension of the subject, wherein
the processor is programmed to select the body outer shape image corresponding to the dimension of the subject.

16. The endoscope insertion shape observation apparatus according to claim 1, wherein the processor is programmed to:
display a viewpoint display indicating a direction that the insertion shape image representing the insertion shape of the insertion section is viewed.

17. The endoscope insertion shape observation apparatus according to claim 1, wherein the processor is programmed to:
multi-screen display a plurality of the insertion shape images and body outer shape images which differ in viewpoints.
